(19) United States
(12) Patent Application Publication
(10) Pub. No.: US 2022/0401936 A1
(43) Pub. Date: Dec. 22, 2022

(54) MOF CATALYSTS FOR OLIGOMERIZATION OF OLEFINS

(71) Applicant: King Abdullah University of Science and Technology, Thuwal (SA)

(72) Inventors: Yu HAN, Thuwal (SA); Xinglong DONG, Thuwal (SA); Mohammed ALALOUNI, Thuwal (SA); Cailing CHEN, Thuwal (SA)

(21) Appl. No.: 17/641,611

(22) PCT Filed: Sep. 8, 2020
(86) PCT No.: PCT/IB2020/058344

§ 371 Date: Mar. 9, 2022

Related U.S. Application Data
(60) Provisional application No. 62/897,611, filed on Sep. 9, 2019.
(60) Provisional application No. 63/026,928, filed on May 19, 2020.

(51) Int. Cl. B01J 31/16; B01J 31/22; C07C 2/32

(52) U.S. Cl. CPC B01J 31/1691; B01J 31/2273; C07C 2/32; C07C 2531/18; B01J 2231/20

(57) ABSTRACT

The present invention encompasses a catalyst composition that includes a heterogeneous oligomerization catalyst including a metal-organic framework, the metal-organic framework including a plurality of first metal ions coordinated to one or more ligands, wherein each of the one or more ligands has only one N-heterocyclic aromatic group. The present invention further includes a method of oligomerization that comprises contacting one or more olefins with the heterogeneous oligomerization catalyst to form one or more oligomers, wherein the heterogeneous catalyst comprises the said metal-organic framework and an optional support.

### BACKGROUND

Various commercialized technologies are employed to produce hydrocarbons. These technologies utilize a homogenous catalyst for the oligomerization of olefins to produce hydrocarbons. Conventional systems based on these technologies have very high catalyst recovery costs because, for example, at least the products and catalysts are in the same phase. In addition, these systems utilize excessively large amounts of organic solvents and thus are not an environmentally friendly option.

### SUMMARY

Metal-organic frameworks that can be utilized as heterogeneous catalysts for olefin oligomerization and/or olefin dimerization, methods of utilizing said metal-organic frameworks for olefin oligomerization and/or olefin dimerization, and the like are disclosed herein.

According to one or more aspects of the invention, a catalyst composition may include a heterogeneous oligomerization catalyst including a metal-organic framework, the metal-organic framework including a plurality of first metal ions coordinated to one or more ligands, wherein each of the one or more ligands has only one N-heterocyclic aromatic group.

According to one or more further aspects of the invention, a method of oligomerization may include contacting one or more olefins with a heterogeneous oligomerization catalyst to form one or more oligomers, wherein the heterogeneous catalyst comprises a metal-organic framework and an optional support, the metal-organic framework including a plurality of first metal ions coordinated to one or more ligands, wherein each of the one or more ligands has only one N-heterocyclic aromatic group.

The details of one or more examples are set forth in the description below. Other features, objects, and advantages will be apparent from the description and from the claims.

### BRIEF DESCRIPTION OF DRAWINGS

This written disclosure describes illustrative embodiments that are non-limiting and non-exhaustive. In the drawings, which are not necessarily drawn to scale, like numerals describe substantially similar components throughout the several views. Like numerals having different letter suffixes represent different instances of substantially similar components. The drawings illustrate generally, by way of example, but not by way of limitation, various embodiments discussed in the present document.

Reference is made to illustrative embodiments that are depicted in the figures, in which:

FIG. 1 is a flowchart of a method of oligomerization, according to one or more embodiments of the present disclosure.

FIG. 2 is a representation of one modeled structure of a metal-doped metal-organic framework heterogeneous catalyst, according to one or more embodiments of the present disclosure.

FIG. 3 is an example of Powder X-ray Diffraction (PXRD) pattern of a metal-doped metal organic framework heterogeneous catalyst, according to one or more embodiments of the present disclosure.

FIG. 4 is an example of a Transmission Electron Microscopy image of a metal-doped metal-organic framework heterogeneous catalyst, according to one or more embodiments of the present disclosure.

FIG. 5 is an example of Energy Dispersive X-ray Spectroscopy images of a metal-doped metal organic framework heterogeneous catalyst, according to one or more embodiments of the present disclosure.

FIG. 6 is an example of a Gas Chromatography record before ethylene dimerization reaction over a metal-doped metal-organic framework heterogeneous catalyst, according to one or more embodiments of the present disclosure.

FIG. 7 is an example of a Gas Chromatography record after ethylene dimerization reaction over a metal-doped metal-organic framework heterogeneous catalyst, according to one or more embodiments of the present disclosure.

FIG. 8 is an example of a Gas Chromatography record after ethylene dimerization reaction over a metal-doped metal-organic framework heterogeneous catalyst, according to one or more embodiments of the present disclosure.

FIG. 9 is an example of a Gas Chromatography record after ethylene dimerization reaction over a metal-doped metal-organic framework heterogeneous catalyst, according to one or more embodiments of the present disclosure.

FIG. 10 is an example of a Gas Chromatography record after ethylene dimerization reaction over a metal-doped metal-organic framework heterogeneous catalyst, according to one or more embodiments of the present disclosure.

FIG. 11 is an example of a Gas Chromatography record after ethylene dimerization reaction over a metal-doped metal-organic framework heterogeneous catalyst, according to one or more embodiments of the present disclosure.

FIG. 12 is an example of a Gas Chromatography record after ethylene dimerization reaction over a metal-doped metal-organic framework heterogeneous catalyst, according to one or more embodiments of the present disclosure.

FIG. 13 is an example of a Gas Chromatography record after ethylene dimerization reaction over a metal-doped metal-organic framework heterogeneous catalyst, according to one or more embodiments of the present disclosure.

FIG. 14 is an example of a Gas Chromatography record after ethylene dimerization reaction over a metal-doped metal-organic framework heterogeneous catalyst, according to one or more embodiments of the present disclosure, according to one or more embodiments of the present disclosure.

FIG. 15 is a schematic representation of a modeled structure of a metal-doped metal-organic framework heterogeneous catalyst, according to one or more embodiments of the present disclosure.

FIG. 16 is a Powder X-ray Diffraction (PXRD) pattern of a metal-doped metal-organic framework heterogeneous catalyst, according to one or more embodiments of the present disclosure.

FIG. 17 is a Scanning Transmission Electron Microscopy (STEM) image of a metal-doped metal-organic framework heterogeneous catalyst, according to one or more embodiments of the present disclosure.

FIG. 18 is an Energy Dispersive X-ray Spectroscopy image of a metal-doped metal-organic framework heterogeneous catalyst, according to one or more embodiments of the present disclosure.

FIG. 19 is a Gas Chromatography record after ethylene oligomerization reaction over a metal-doped metal-organic framework heterogeneous catalyst, according to one or more embodiments of the present disclosure.

FIG. 20 is a schematic representation of a modeled structure of a metal-doped metal-organic framework heterogeneous catalyst, according to one or more embodiments of the present disclosure.

FIG. 21 is a Powder X-ray Diffraction (PXRD) pattern of a metal-doped metal-organic framework heterogeneous catalyst, according to one or more embodiments of the present disclosure.

FIG. 22 is a Scanning Transmission Electron Microscopy (STEM) image of a metal-doped metal-organic framework heterogeneous catalyst, according to one or more embodiments of the present disclosure.

FIG. 23 is an Energy Dispersive X-ray Spectroscopy image of a metal-doped metal-organic framework heterogeneous catalyst, according to one or more embodiments of the present disclosure.

FIG. 24 is a Gas Chromatography record after propylene dimerization reaction over a metal-doped metal-organic framework heterogeneous catalyst, according to one or more embodiments of the present disclosure.

### DETAILED DESCRIPTION

The present invention relates to metal-organic frameworks and their use as heterogeneous catalysts, methods of utilizing the metal-organic frameworks as heterogeneous catalysts, and the like. It was surprisingly discovered that the metal-organic frameworks disclosed herein perform exceptionally well as heterogeneous catalysts for the oligomerization of olefins. These heterogeneous oligomerization catalysts are superior to conventional catalysts in numerous ways. For example, the heterogenous catalysts can produce one or more oligomers, as reaction products, with very high selectivity, while exhibiting a catalytic activity that is 20 times the catalytic activity of conventional heterogenous catalysts. In addition, the heterogeneity of the metal-organic frameworks is advantageous because, following the oligomerization, the heterogeneous catalysts disclosed herein can be readily and easily recovered, recycled, and/or re-used in one or more subsequent reaction cycles. Accordingly, the costly processes required to separate conventional catalysts from reaction products can be avoided.

In addition, the catalytic performance of the heterogeneous catalysts disclosed herein is superior to conventional catalysts in numerous ways. For example, in some embodiments, the heterogeneous catalysts can be used to produce one or more butenes, including 1-butene, via the dimerization of ethylene, with a selectivity of at least 90% and an activity or turnover frequency of at least about 120,000 moles of ethylene per mole of active metal per hour. In further embodiments, the heterogeneous catalysts can be used to produce one or more C₆ hydrocarbons, via the dimerization of propylene, with a selectivity of at least 90% and a production rate of at least 2700 grams of products per gram of catalyst. In still further embodiments, the heterogeneous catalysts can be used to produce wide range linear alpha olefins, via the oligomerization of ethylene, with a selectivity of at least 60% and a production rate of at least 100 grams of products per gram of catalyst. These and other advantages are provided below and elsewhere throughout the present disclosure.

### Definitions

The terms recited below have been defined as described below. All other terms and phrases in this disclosure shall be construed according to their ordinary meaning as understood by one of skill in the art.

Unless otherwise provided, all percentages by weight are based on total weight of the metal-organic frameworks and/or heterogeneous catalysts. Weight percent can be measured using Inductively Coupled Plasma (ICP), X-ray Fluorescence (XRF), or other techniques known in the art.

As used herein, the term “homogeneous catalyst” refers to a catalyst that is present in the same phase as reactants and/or products.

As used herein, the term “heterogeneous catalyst” refers to a catalyst that is present in a phase that is different from the phase of reactants and/or products.

As used herein, the “turnover frequency” or “TOF” is a unit of measure that refers to the total number of moles transformed into the desired product by one mole of active site per hour.

As used herein, the term “selectivity” refers to a percentage of reactant converted to a specified product. For example, in reactions in which butene is produced by ethylene dimerization, the selectivity can refer to the percentage of ethylene that is converted to butenes generally or a specific butene, such as 1-butene.

As used herein, the term “oligomerization” refers to any reaction involving the formation of one or more oligomers from one or more monomers. The term includes dimerization reactions, trimerization reactions, tetramerization reactions, pentamerization reactions, and so on. As used herein, the term “dimerization” generally refers to an organic addition reaction in which two reactant molecules (e.g., monomers) combine through covalent or intermolecular bonds to produce dimers. Similarly, a trimerization reaction refers to an organic additional reaction in which three reactant molecules (e.g., monomers) combine through covalent or intermolecular bonds to produce trimers. Tetramerization reactions, pentamerization reactions, etc. refer to similar reactions, except additional reactant molecules are combined. As used herein, the term “oligomer” refers to any molecule including one or more repeat units. For example, the term includes a molecule comprising between 2 to 50 repeat units. Non-limiting examples of oligomers include dimers, trimers, tetramers, and pentamers, among others.

As used herein, the term “olefin” refers to any hydrocarbon containing at least one carbon-carbon double bond. Olefins can be used as monomers and/or formed as reaction products. Non-limiting examples of olefins include alkenes including two or more carbon atoms, such as ethylene, propylene, butene, alpha olefins, linear alpha olefins, and the like. The term “Cₙ olefin” refers to an olefin as defined herein that includes “n” carbon atoms. For example, a C₄ olefin refers to an olefin containing 4 carbon atoms (e.g., butenes, such as 1-butene, 2-butene, isobutene, etc.).

As used herein, the term “linear alpha olefin” refers to alkenes with linear carbon chains and a double bond at an alpha position. Linear alpha olefins can be represented, for example, by the chemical formula: CH₂═CH—(CH₂)ₓ—CH₃, where x is 0 or higher. The term “Cₙ linear alpha olefins” generally refers to a linear alpha olefin having a carbon chain-length of “n.” For example, a C₆ linear alpha olefin includes a linear alpha olefin having a chain-length of 6 carbon atoms. A C₄-C₁₀ linear alpha olefin includes linear alpha olefins having a chain-length of 4 to 10 carbon atoms. In general, the carbon chain-length of linear alpha olefins can range from 3 carbon atoms or greater (e.g., 30-plus carbon atoms).

As used herein, the term “transition metal” refers to elements in the d-block and f-block of the Period Table of Elements. Accordingly, the term “transition metal” includes metals in Groups IIIB to IIB, as well as lanthanoids and actinoids, which collectively can be referred to as “inner transition metals.”

As used herein, the term “ligand” refers to any chemical species that can bind to a metal, a metal ion, and/or a cluster containing at least one metal and/or at least one metal ion. Ligands usually have at least one, more frequently at least two, binding sites available for linking or coordinating to metals and/or metal ions (e.g., via a coordinate bond). Ligands having two or more binding sites can be referred to as polytopic or polydentate ligands. The ligands referred to herein include mono- and polytopic ligands.

As used herein, the term “N-heterocyclic aromatic group” refers to an aromatic group comprising at least one nitrogen atom, wherein the at least one nitrogen atom forms part of an aromatic ring structure in which at least one of the ring atoms is nitrogen. The aromatic ring structure can include three or more ring atoms (e.g., from 3 to 100 ring atoms), and one or more of the ring atoms can be a nitrogen atom.

The N-heterocyclic aromatic groups can comprise one or more rings. If more than one ring is present, the rings may be fused or not fused, for example, bridged. The N-heterocyclic aromatic groups can have 30 or fewer carbon atoms in the aromatic ring structure, 20 or fewer carbon atoms, 15 or fewer carbon atoms, 10 or fewer carbon atoms; and 10 or fewer nitrogen heteroatoms, 5 or fewer nitrogen heteroatoms, 3 or fewer nitrogen heteroatoms. The term does not preclude the presence of heteroatoms other than nitrogen, nor does it preclude the presence of nitrogen atoms in positions outside of the aromatic ring structure. The N-heterocyclic aromatic groups can be substituted or unsubstituted.

As used herein, “heteroatom” means an atom of any element other than carbon or hydrogen. Non-limiting examples of heteroatoms include nitrogen, oxygen, and sulfur. As discussed herein, heteroatoms, such as nitrogen, may have hydrogen substituents and/or any permissible substituents of organic compounds described herein which satisfy the valences of the heteroatoms.

As used herein, the term “alkyl” refers to an alkane with at least one hydrogen atom removed and includes, without limitation, straight chain alkyl groups, branched chain alkyl groups, cycloalkyl (alicyclic) groups, alkyl-substituted cycloalkyl groups, and cycloalkyl-substituted alkyl groups. A straight chain or branched chain alkyl has 30 or fewer carbon atoms in its backbone (e.g., C₁-C₃₀ for straight chains, and C₃-C₃₀ for branched chains). Cycloalkyls have 3-10 carbon atoms in their ring, preferably 5-6 carbons in the ring. The groups —CH₃ (Me), —CH₂CH₃ (Et), —CH₂CH₂CH₃ (n-Pr), —CH(CH₃)₂ (iso-Pr), —CH(CH₂)₂ (cyclopropyl), —CH₂CH₂CH₂CH₃ (n-Bu), —CH(CH₃)CH₂CH₃ (sec-butyl), —CH₂CH(CH₃)₂ (iso-butyl), —C(CH₃)₃ (tert-butyl), —CH₂C(CH₃)₃ (neo-pentyl), cyclopropyl, cyclobutyl, cyclopentyl, cyclohexyl, and cyclohexylmethyl are non-limiting examples of alkyl groups. Additional examples of alkyl groups include, but are not limited to, methyl, ethyl, 1-propyl, 2-propyl, 1-butyl, 2-butyl, 2-methyl-1-propyl, 2-methyl-2-propyl, 1-pentyl, 2-pentyl, 3-pentyl, 2-methyl-1-butyl, 3-methyl-1-butyl, 2-methyl-3-butyl, 2,2-dimethyl-1-propyl, 1-hexyl, 2-hexyl, 3-hexyl, 2-methyl-1-pentyl, 3-methyl-1-pentyl, 4-methyl-1-pentyl, 2-methyl-2-pentyl, 3-methyl-2-pentyl, 4-methyl-2-pentyl, 2,2-dimethyl-1-butyl, 3,3-dimethyl-1-butyl and 2-ethyl-1-butyl, 1-heptyl, and 1-octyl. Alkyls can optionally be substituted. When the term is used with the “substituted” modifier, one or more hydrogen atoms has been independently replaced by any of the substituents disclosed herein.

As used herein, the term “aliphatic” refers to acyclic or cyclic, but non-aromatic hydrocarbon compounds or groups. In aliphatic compounds/groups, the carbon atoms can be joined together in straight chains, branched chains, or non-aromatic rings (alicyclic). Aliphatic compounds/groups can be saturated, that is joined by single bonds (alkanes/alkyl), or unsaturated, with one or more double bonds (alkenes/alkenyl) or with one or more triple bonds (alkynes/alkynyl).

As used herein, the term “aromatic” refers to a planar ring having a delocalized π-electron system containing 4n+2 π-electrons, where n is an integer. Aromatic rings can be formed by five, six, seven, eight, nine, or more than nine atoms. Aromatics can be optionally substituted. The term “aromatic” includes both carbocyclic aryl (e.g., phenyl) and heterocyclic aryl (or “heteroaryl” or “heteroaromatic”) groups (e.g., pyridine). The term also includes monocyclic or fused-ring polycyclic (i.e., rings which share adjacent pairs of carbon atoms) groups.

As used herein, the term “aryl” refers to a monocyclic or polycyclic aromatic hydrocarbon radical or moiety comprising only carbon and hydrogen atoms, wherein the carbon atoms form an aromatic ring structure. If more than one ring is present, the rings may be fused or not fused, or bridged. The term does not preclude the presence of one or more alkyl groups attached to the first aromatic ring or any additional aromatic ring present. The point of attachment can be through an aromatic carbon atom in the ring structure or a carbon atom of an alkyl group attached to the ring structure. Non-limiting examples of aryl groups include phenyl (Ph), toyl, xylyl, methylphenyl, (dimethyl)phenyl, —C₆H₄—CH₂CH₃ (ethylphenyl), naphthyl, and the monovalent group derived from biphenyl. Further examples of aryl groups having only aromatic carbocyclic ring(s) include phenyl, 1-naphthyl (bicyclic), 2-naphthyl (bicyclic), anthracenyl (tricyclic), phenanthrenyl (tricyclic), pentacenyl (pentacyclic), and like groups. Examples of polycyclic ring systems in which at least one aromatic carbocyclic ring is fused to one or more cycloalkyl and/or cycloheteroalkyl rings include, among others, benzo derivatives of cyclopentane (i.e., an indanyl group, which is a 5,6-bicyclic cycloalkyl/aromatic ring system), cyclohexane (i.e., a tetrahydronaphthyl group, which is a 6,6-bicyclic cycloalkyl/aromatic ring system), imidazoline (i.e., a benzimidazolinyl group, which is a 5,6-bicyclic cycloheteroalkyl/aromatic ring system), and pyran (i.e., a chromenyl group, which is a 6,6-bicyclic cycloheteroalkyl/aromatic ring system). Other examples of aryl groups include benzodioxanyl, benzodioxolyl, chromanyl, indolinyl groups, and the like. When the term is used with the “substituted” modifier, one or more hydrogen atoms has been independently replaced by any of the substituents disclosed herein.

As used herein, the term “heteroaryl” refers to an aryl having at least one aromatic carbon atom in the ring structure replaced by a heteroatom. Non-limiting examples of suitable heteroatoms include nitrogen, oxygen, and sulfur. The term does not preclude the presence of one or more alkyl groups attached to the first aromatic ring or any additional aromatic ring present. The point of attachment can be through an aromatic carbon atom or aromatic heteroatom in the aromatic ring structure or a carbon atom of an alkyl group attached to the aromatic ring structure. Non-limiting examples of heteroaryl groups include furanyl, benzofuranyl, isobenzylfuranyl, imidazolyl, indolyl, isoindolyl, indazolyl, methylpyridyl, oxazolyl, pyridyl, pyrrolyl, pyrimidyl, pyrazinyl, quinolyl, quinazolyl, quinoxalinyl, thienyl, and triazinyl. Additional examples of heteroaryl groups include, for example, the 5- or 6-membered monocyclic and 5-6 bicyclic ring systems shown below:

As used herein, the term “substituted” refers to all permissible substituents of the compounds described herein. In the broadest sense, the permissible substituents include acyclic and cyclic, branched and unbranched, carbocyclic and heterocyclic, aromatic and nonaromatic substituents of organic compounds. Non-limiting examples of substituents include halogens, hydroxyl groups, or any other organic groupings containing any number of carbon atoms and optionally include one or more heteroatoms such as oxygen, nitrogen, or sulfur grouping in linear, branched, or cyclic structural formats.

Representative substituents include halo, hydroxyl, alkyl, substituted alkyl, alkenyl, substituted alkenyl, alkynyl, substituted alkynyl, phenyl, substituted phenyl, aryl, substituted aryl, heteroaryl, substituted heteroaryl, alkoxy, substituted alkoxy, phenoxy, substituted phenoxy, aryloxy, substituted aryloxy, aralkoxy, substituted aralkoxy, alkenyloxy, substituted alkenyloxy, alkynyloxy, substituted alkynyloxy, heteroaryloxy, substituted heteroaryloxy, acyloxy, substituted acyloxy, carbonyl, substituted carbonyl, carboxyl, substituted carboxyl, amino, substituted amino, amido, substituted amido, sulfonyl, substituted sulfonyl, arylsulfonyl, substituted arylsulfonyl, sulfonic acid, phosphoryl, substituted phosphoryl, phosphonyl, substituted phosphonyl, polyaryl, substituted polyaryl, C₃-C₂₀ cyclic, substituted C₃-C₂₀ cyclic, heterocyclic, substituted heterocyclic, and amino acid groups.

As used herein, the term “bite angle” refers to a bond angle of a metal-organic framework. Examples of bond angles include, but are not limited to, ligand-metal-ligand bond angles, ligand-metal-counterion (and/or electron donor) bond angles, and the like. A specific example of a bite angle is a N-M-A bond angle, where N is a nitrogen atom, M is a metal, and A is a counterion (and/or electron donor) (e.g., halides such as Cl, Br, I, etc.; anions such as NO₃⁻, etc.; electron donors such as 1,5-Cyclooctadiene, etc.) Another specific example of a bite angle is a N-M-L bond angle, where N and M are as defined above, and L is a ligand.

As used herein, the term “dopant” is defined broadly and refers to any species associated with a metal-organic framework. The dopant or dopant species is typically a metal ion. The presence of a dopant is not required and thus optional. The dopant species is usually present in a non-stoichiometric amount, but in some instances, species present in a stoichiometric amount can be referred to as dopants. The techniques or processes used to introduce a dopant are not particularly limited. Examples of techniques or processes for introducing a dopant include, but are not limited to, wetness impregnation, ion exchange, hydrothermal synthesis, one-pot synthesis, post-modification, pyrolysis, and other techniques known in the art. The dopant can be incorporated or integrated into the framework of the metal-organic framework or not. For example, the dopant species can be coordinated to a ligand, or the dopant species can be deposited within a pore and/or on a surface of a metal-organic framework. Another example is where a first metal ion is exchanged with or replaced by a metal dopant (e.g., second metal ion).

### Heterogeneous Catalysts Based on Metal-Organic Frameworks

Metal-organic frameworks that can be utilized as heterogeneous catalysts for the oligomerization of one or more olefins are disclosed herein. The metal-organic frameworks can include one or more first metal ions coordinated to one or more ligands, wherein each of the one or more ligands has only one N-heterocyclic aromatic group. The one or more first metal ions can be coordinated to the one or more ligands through, for example, a nitrogen heteroatom of the N-heterocyclic aromatic group or through another coordinating moiety, such as O- and/or S-donor functional groups. In some embodiments, the metal-organic frameworks further comprise one or more metal dopants. For example, the metal-organic frameworks can optionally further comprise one or more second metal ions. The one or more second metal ions can be integrated into the metal-organic framework and/or deposited on the surface and/or pores of the metal-organic framework, depending on the technique used to introduce the one or more second metal ions, among other things. Where integrated (e.g., where a second metal ion replaces a first metal ion), one or more second metal ions can be coordinated to one or more ligands in the same or similar manner as the one or more first metal ions.

In some embodiments, the metal-organic framework is supported on a substrate or other support material. Such supported heterogeneous catalysts can be desirable in instances where the catalyst size is small (e.g., approximately less than about 50 nm, among other dimension lengths). By loading the catalyst onto a support or substrate, collection and use of the catalysts is more convenient and practical. Other benefits of utilizing a support include, but are not limited to, preventing or reducing the likelihood of sintering or agglomeration which can lead to deactivation of the catalytic sites, as well as providing, at least from a commercial or industrial perspective, a regular array of well-defined about equally-sized and similarly-shaped catalysts which can impart mechanical stability and make them easier to process and manufacture, among other things. The supports are not particularly limited. An example of a suitable support includes inert supports, such as carbon, silica, polymers, monoliths, and clays. Other supports can be utilized without departing from the scope of the present disclosure.

In some embodiments, the metal-organic frameworks can be characterized as monometallic heterogeneous catalysts and/or bimetallic heterogeneous catalysts. For example, in some embodiments, the metal-organic frameworks include one or more first metal ions and optionally one or more second metal ions. In some embodiments, the one or more first metal ions and/or, if present, the one or more second metal ions of the metal-organic frameworks can provide one or more of the active sites for catalysis and/or the porous structure. In some embodiments, the one or more first metal ions provide both the active sites for catalysis and the porous structure, optionally without having any second metal ions integrated and/or associated with the metal-organic framework. In some embodiments, the one or more first metal ions and the one or more second metal ions provide the active sites for catalysis, and at least one of said first metal ions and said second metal ions provide the porous structure. In some embodiments, the first metal ions provide the porous structure, but otherwise are at least somewhat inert or inactive. In some of these embodiments, it may be desirable to introduce a metal dopant to provide the active sites for catalysis. Accordingly, in some embodiments, the one or more first metal ions can provide the porous structure and the one or more second metal ions can provide the active sites for catalysis. Other variations can be employed without departing from the scope of the present disclosure.

The metal-organic framework can include one or more first metal ions. In some embodiments, the metal-organic framework includes a plurality of first metal ions. In some embodiments, each first metal ion of the plurality of first metal ions is coordinated to one or more ligands. In some embodiments, the metal-organic framework includes a plurality of clusters, wherein each cluster contains one or more first metal ions. In some embodiments, each cluster of the plurality of clusters is coordinated to one or more ligands. The one or more first metal ions can include and/or can be selected from any transition metal. As used herein, the term “transition metal” generally refers to metals in Groups IIIB to IIB of the Periodic Table of Elements, as well as lanthanoids and actinoids. Examples of suitable transition metals include, but are not limited to, zinc, titanium, nickel, copper, chromium, vanadium, cadmium, ruthenium, manganese, iron, cobalt, zirconium, silver, gold, magnesium, palladium, platinum, mercury, lead, aluminum, scandium, indium, and gallium. The valence of the transition metals, as first metals and/or first metal ions, is not particularly limited and can include zerovalent metals, monovalent metal ions, divalent metal ions, trivalent metal ions, tetravalent metal ions, pentavalent metal ions, and so on. Examples of suitable mono- and multi-valent transition metal ions include, but are not limited to, Zn²⁺, Ti³⁺, Ti⁴⁺, Ni²⁺, Cu²⁺, Cr²⁺, Cr³⁺, V²⁺, V³⁺, Cd²⁺, Ru²⁺, Mn²⁺, Fe³⁺, Fe²⁺, Co²⁺, Ag⁺, Cu⁺, Au⁺, Mg²⁺, Pd²⁺, Pt²⁺, Pb²⁺, Hg²⁺, Sc³⁺, Al³⁺, In³⁺, Ga³⁺, Mn³⁺, Zr⁺¹, Zr⁺², Zr⁺³, Zr⁺⁴, and Co³⁺.

The metal-organic framework can include a metal dopant, wherein the metal dopant includes one or more second metal ions. The one or more second metal ions can be deposited on the surface and/or pores of the metal-organic framework, or the one or more second metal ions can be integrated into the metal-organic framework as described above (e.g., in the same or similar manner as the one or more first metal ions). The one or more second metal ions, if present, usually comprise at least one metal that is different from the one or more first metal ions. The one or more second metal ions can include and/or can be selected from any transition metal as defined herein. Examples of suitable transition metals include, but are not limited to, zinc, titanium, nickel, copper, chromium, vanadium, cadmium, ruthenium, manganese, iron, zirconium, cobalt, silver, gold, magnesium, palladium, platinum, mercury, lead, aluminum, scandium, indium, and gallium. The valence of the transition metals, as second metals and/or second metal ions, is not particularly limited and can include zerovalent metals, monovalent metal ions, divalent metal ions, trivalent metal ions, tetravalent metal ions, and so on. Examples of suitable mono- and multi-valent transition metal ions include, but are not limited to, Zn²⁺, Ti³⁺, Ti⁴⁺, Ni²⁺, Cu²⁺, Cr²⁺, Cr³⁺, V²⁺, V³⁺, Cd²⁺, Ru²⁺, Mn²⁺, Fe³⁺, Fe²⁺, Co²⁺, Ag⁺, Cu⁺, Au⁺, Mg²⁺, Pd²⁺, Pt²⁺, Pb²⁺, Hg²⁺, Sc³⁺, Al³⁺, In³⁺, Ga³⁺, Mn³⁺, Zr⁺¹, Zr⁺², Zr⁺³, Zr⁺⁴, and Co³⁺.

In certain embodiments, the one or more first metals ions include one or more of titanium, nickel, copper, chromium, and vanadium. In certain embodiments, the one or more first metal ions are selected from the group consisting of Zn²⁺, Ti³⁺, Ti⁴⁺, Ni²⁺, Cu²⁺, Cr²⁺, Cr³⁺, V²⁺, V³⁺, Cd²⁺, or Ru²⁺. In certain embodiments, the one or more first metal ions are selected from the group consisting of Ti³⁺, Ti⁴⁺, Ni²⁺, Cu²⁺, Cr²⁺, Cr³⁺, V²⁺, V³⁺, or Cd²⁺. In certain embodiments, the one or more second metals include one or more of titanium, nickel, copper, chromium, and vanadium. In certain embodiments, the one or more second metal ions are selected from the group consisting of Ti³⁺, Ti⁴⁺, Ni²⁺, Cu²⁺, Cr²⁺, Cr³⁺, V²⁺, or V³⁺. In certain embodiments, the one or more first metal ions include zinc (e.g., Zn²⁺) and the one or more second metals include nickel (e.g., Ni²⁺). In certain embodiments, the one or more first metal ions include cobalt (e.g., Co²⁺ or Co³⁺ or both) and the one or more second metal ions include nickel (e.g., Ni²⁺). These shall not be limiting as any combination of transition metals of any valence can be used herein without departing from the scope of the present disclosure.

The first metal ion content of the metal-organic frameworks can be in the range of about 0.001% to about 60% by weight. In some embodiments, the first metal ion content of the metal-organic frameworks is less than or about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, or any increment thereof. In certain embodiments, the first metal ion content of the metal-organic frameworks is up to about 30%, up to about 20% by weight, up to about 15% by weight, up to about 14% by weight, up to about 13% by weight, up to about 12% by weight, up to about 11% by weight, up to about 10% by weight, up to about 9% by weight, up to about 8% by weight, up to about 7% by weight, up to about 6% by weight, up to about 5% by weight, up to about 4% by weight, up to about 3% by weight, up to about 2% by weight, up to about 1% by weight, or any increment thereof. In certain embodiments, the first metal ion content of the metal-organic frameworks is in the range of about 20% to about 30% by weight. In certain embodiments, the first metal ion content of the metal-organic frameworks is less than about 5% or even less than about 1% by weight, wherein the weight percentage is based on total weight of the catalyst including the support.

As described above, the one or more second metal ions is optional. If present, the second metal ion content of the metal-organic frameworks can be in the range of about 0% to about 60% by weight. In certain embodiments, the second metal ion content of the metal-organic frameworks is about 0% by weight, about 1% by weight, about 5% by weight, about 10% by weight, about 15% by weight, about 20% by weight, about 25% by weight, about 30% by weight, about 35% by weight, about 40% by weight, about 45% by weight, about 50% by weight, about 55% by weight, about 60% by weight, or any increment thereof. In certain embodiments, the second metal ion content of the metal-organic frameworks is up to about 20% by weight, up to about 15% by weight, up to about 14% by weight, up to about 13% by weight, up to about 12% by weight, up to about 11% by weight, up to about 10% by weight, up to about 9% by weight, up to about 8% by weight, up to about 7% by weight, up to about 6% by weight, up to about 5% by weight, up to about 4% by weight, up to about 3% by weight, up to about 2% by weight, up to about 1% by weight, or 0% by weight, or any increment thereof. In certain embodiments, the second metal ion content of the metal-organic frameworks is in the range of about 0.1% to about 10% by weight. In certain embodiments, the second metal ion content of the metal-organic frameworks is less than about 5% or even less than about 1% by weight, wherein the weight percentage is based on total weight of the catalyst including the support.

In some embodiments, each of the one or more ligands has only one N-heterocyclic aromatic group. In some embodiments, the heterogeneous catalyst and thus the metal-organic framework only includes ligands having one N-heterocyclic aromatic group. For example, in some embodiments, the heterogeneous catalyst, including the metal-organic framework includes one or more ligands, wherein the one or more ligands consist of ligands having only one N-heterocyclic aromatic group. The N-heterocyclic aromatic group can comprise a single aromatic ring structure including one or more nitrogen heteroatoms as ring atoms. In some embodiments, the single aromatic ring structure comprising one or more nitrogen heteroatoms is a 5- or 6-membered ring. For example, in some embodiments, the single aromatic ring structure is a 5-membered ring comprising one nitrogen heteroatom, two nitrogen heteroatoms, three nitrogen heteroatoms, or four nitrogen heteroatoms. The aromatic ring structure can be substituted or unsubstituted, and the other atoms in the aromatic ring structure can be carbon atoms and/or heteroatoms other than nitrogen (e.g., S atoms, 0 atoms, etc.). In some embodiments, the single aromatic ring structure is a 6-membered ring comprising one nitrogen heteroatom, two nitrogen heteroatoms, three nitrogen heteroatoms, four nitrogen heteroatoms, or five nitrogen heteroatoms. The aromatic ring structure can be substituted or unsubstituted, and the other atoms in the aromatic ring structure can be carbon atoms and/or heteroatoms other than nitrogen (e.g., S atoms, 0 atoms, etc.). Examples of N-heterocyclic aromatic groups include, but are not limited to, pyrrolate, pyrazolate, triazolate, imidazolate, oxazolate, tetrazolate, pyridinate, thiazolate, oxadiazolate, purinate, quinolonate, and indolate, any of which can be substituted or unsubstituted.

In some embodiments, the single aromatic ring structures described above can optionally be fused, bridged, and/or attached to one or more substituted and/or unsubstituted cyclic rings. For example, in some embodiments, a single aromatic ring structure is fused and/or attached to one or more substituted and/or unsubstituted rings. In some embodiments, the one or more substituted and/or unsubstituted rings include or only include rings other than an N-heterocyclic aromatic group. The one or more substituted and/or unsubstituted rings each can have from three to fifty ring atoms, each can be aromatic or alicyclic, and/or each can comprise one or more carbon atoms and/or one or more heteroatoms. For example, in some embodiments, the N-heterocyclic group is fused or attached to one or more carbocyclic and/or heterocyclic rings. In addition or in the alternative, the single aromatic ring structure can optionally be attached to one or more acyclic compounds, which can be saturated or unsaturated, linear or branched, substituted or unsubstituted. The fused rings, attached rings, and/or attached acyclic compounds can optionally be fused or attached to additional rings and/or acyclic compounds with the same or similar characteristics. In some embodiments, rings, either fused or attached, and acyclic compounds comprise a coordinating S-donor and/or 0-donor functional group.

In certain embodiments, the metal-organic frameworks comprise one or more ligands of formulas (I) and/or (II):

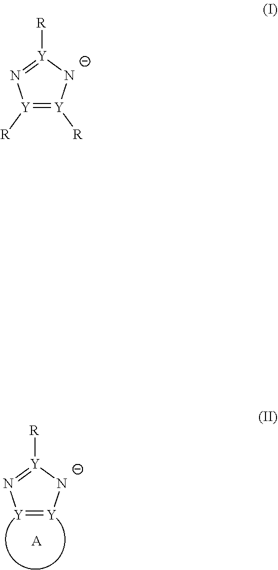

wherein each Y is independently N or C; each R is the same or different and each is independently selected from the group consisting of nothing, hydrogen, -alkyl, and counterion (e.g., halides, such as Cl, Br, I, and F; etc.); A is an aromatic or aliphatic substituent group (e.g., a fused aromatic group, a fused aliphatic group, etc.) optionally without coordinating sites (e.g., benzene). Each R can independently be substituted or unsubstituted. A can be substituted or unsubstituted. In some embodiments, the metal-organic frameworks comprise one or more ligands of the formula: R^{a}—(N-heterocyclic aromatic group)-R^{b}, wherein R^{a} and R^{b} can be the same or different and each of R^{a} and R^{b} is independently selected from the group consisting of: hydrogen, -alkyl, —NO₂, —R′, —F, —Cl, —Br, —I, —CN, —NC, —SO₃R′, —SO₃H, —OR′, —OH, —SR′, —SH, —PO₃R′, —PO₃H, —CF₃, —NR′2, —NHR′, and —NH₂, wherein each R′ is the same or different and each is optionally substituted alkyl or optionally substituted aryl. In some embodiments, R′ is an optionally substituted aryl, provided that R′ is not an N-heterocyclic aryl.

In some embodiments, the first metal ion(s), second metal ion(s), or both the first and second metal ions (collectively, “metal ions” for simplicity) can associate to the ligand through coordinating N-donor, O-donor, and/or S-donor functional groups. In some embodiments, the coordinating N-donor functional group is the N-heterocyclic aromatic group. For example, in some embodiments, one or more of the first metal ions and, if present, one or more of the second metal ions can be coordinated to a ligand through at least one of the nitrogen heteroatoms of the N-heterocyclic aromatic group. In some embodiments, one or more of the first metal ions and, if present, one or more of the second metal ions can be coordinated to a ligand through an O atom or S atom of the coordinating functional group. Non-limiting examples of coordinating N-, O-, and S-donor functional groups include amides (including sulfonamide and phosphoramides), sulfinic acids, sulfonic acids, phosphonic acids, phosphates, phosphodiesters, phosphines, boronic acids, boronic esters, borinic acids, borinic esters, nitrates, nitrites, nitriles, nitro, nitroso, thiocyanates, cyanates, azos, azides, imides, imines, amines, acetals, ketals, ethers, esters, aldehydes, ketones, alcohols, thiols, sulfides, disulfides, sulfoxides, sulfones, sulfinic acids, thiones, and thials.

The metal-organic frameworks can be characterized by a bite angle. In some embodiments, the metal-organic frameworks can be characterized by a wide bite angle or a bite angle that is wider than conventional catalysts and/or conventional metal-organic frameworks. The bite angle can be characterized by N-M-A bond angles and/or N-M-L bond angles, where N is a nitrogen atom, M is a metal or metal ion (e.g., first metal ion, second metal ion, metal dopant, etc.), A is a counterion or electron donor (e.g., halides, anions, 1,5-cyclooctadiene, etc.), and L is a ligand. The bite angle can be in the range of about 0° to about 360°, or any increment thereof. For example, in certain embodiments, the bite angle can be in the range of about 50° to about 180°. In certain embodiments, the bite angle is about 109°, wherein the bite angle is a N—Ni—NO₃ bond angle or N—Ni-L bond angle, where L is a ligand. In certain embodiments, the bite angle is about 145°. In certain embodiments, the bite angle is an angle other than about 120°. In certain embodiments, the bite angle is an angle other than about 129°. In certain embodiments, the bite angle is an angle other than about 97°. In certain embodiments, the bite angle is an angle other than about 93°. In certain embodiments, the bite angle can be an angle other than any angle in the range of about 0° to about 360°.

The average particle size of the heterogeneous catalyst or metal-organic frameworks can be in the range of about 5 nm to about 5,000,000 nm, or any increment thereof. For example, in some embodiments, the average particle size of the solid heterogeneous catalysts is in the range of about 5 nm to about 10 nm, about 5 nm to about 50 nm, about 5 nm to about 100 nm, about 5 nm to about 190 nm, 5 nm to about 200 nm, 50 nm to about 100 nm, about 50 nm to about 250 nm, about 250 nm to about 500 nm, about 250 nm to about 750 nm, about 250 nm to about 1000 nm, about 1000 nm to about 1500 nm, about 1000 nm to about 2000 nm, about 2000 nm to about 2500 nm, about 2000 nm to about 3000, about 3000 nm to about 3500 nm, about 3000 nm to about 4000 nm, about 4000 nm to about 4500 nm, about 4000 nm to about 5000 nm, or any increment thereof.

In certain embodiments, the average particle size of the heterogeneous catalysts is less than about 200 nm, less than about 195 nm, less than about 190 nm, less than about 185 nm, less than about 180 nm, less than about 175 nm, less than about 170 nm, less than about 165 nm, less than about 160 nm, less than about 155 nm, less than about 150 nm, less than about 150 nm, less than about 145 nm, less than about 140 nm, less than about 135 nm, less than about 130 nm, less than about 125 nm, less than about 120 nm, less than about 115 nm, less than about 110 nm, less than about 105 nm, less than about 100 nm, less than about 90 nm, less than about 80 nm, less than about 70 nm, less than about 60 nm, less than about 50 nm, less than about 40 nm, less than about 30 nm, less than about 20 nm, less than about 10 nm, less than about 5 nm, or any increment thereof. In certain embodiments, the average particle size of the solid heterogeneous catalysts is an average particle size other than about 150 nm to about 250 nm. In certain embodiments, the average particle size of the solid heterogeneous catalysts is an average particle size other than about 200 nm.

The metal-organic frameworks and/or solid heterogeneous catalysts can be characterized by a temperature stability—for example, the metal-organic frameworks can be stable (e.g., remain catalytically active and/or exhibit minimal or no degradation) at certain temperatures or within certain temperature ranges. For example, the metal-organic frameworks can be utilized as solid heterogeneous catalysts and remain stable at temperatures of at least about 100° C., at least about 125° C., at least about 150° C., at least about 175° C., at least about 200° C., at least about 225° C., at least about 250° C., at least about 275° C., at least about 300° C., at least about 325° C., at least about 350° C., at least about 375° C., at least about 400° C., at least about 425° C. or greater, or any increment thereof. In certain embodiments, the metal-organic frameworks are stable at temperatures of at least about 100° C. In certain embodiments, the metal-organic frameworks are stable at temperatures of at least about 200° C. In certain embodiments, the metal-organic frameworks are stable at temperatures of at least about 400° C.

The metal-organic frameworks can be utilized as catalysts in the oligomerization of olefins and, in particular, the dimerization of ethylene to form, for example, butene (e.g., 1-butene and/or other butenes). The metal-organic frameworks are preferably utilized as heterogeneous catalysts (e.g., solid heterogeneous catalysts), but they can also be utilized as homogeneous catalysts. The catalysts can include or be based on any of the metal-organic frameworks disclosed herein. Accordingly, in some embodiments, heterogeneous catalysts comprising metal-organic frameworks are provided. In some embodiments, metal-organic frameworks are provided, wherein the metal-organic frameworks can be utilized as solid heterogeneous catalysts (e.g., or, in some instances, homogeneous catalysts).

The method of synthesizing the metal-organic frameworks can affect their performance as heterogeneous catalysts, as well as their structural characteristics, among other features. The metal-organic frameworks can be prepared in a simple one-pot synthesis. For example, the metal-organic frameworks can be prepared by contacting or mixing a first metal ion precursor and optionally a second metal ion precursor in a first solvent to form a first solution. The first solution can be contacted and/or mixed with a second solution comprising a ligand precursor and a second solvent, and allowed to react for a select duration, before being centrifuged and washed to obtain the metal-organic framework. The reaction conditions are not particularly limited and can proceed at about atmospheric pressure and/or ambient temperature (e.g., about room temperature). In some embodiments, the metal-organic framework is dried for a select duration.

The amount of the first metal ion precursor and, if present, second metal ion precursor can be varied to adjust or modify the content of the metal-organic framework. In some embodiments, the first and second metal ion precursors are provided in the form of metal salts or metal salt hydrates (e.g., Zn(NO₃)₂.6H₂O, Ni(NO₃)₂.6H₂O, etc.). The solvents are not particularly limited and are general known in the art. One example is methanol. Others can be used without departing from the scope of the present disclosure. Also, the ligand precursor can include any of the ligands disclosed herein. One example of a ligand precursor is 2-methylimidazole. Others can similarly be used without departing from the scope of the present disclosure.

### Methods of Oligomerization

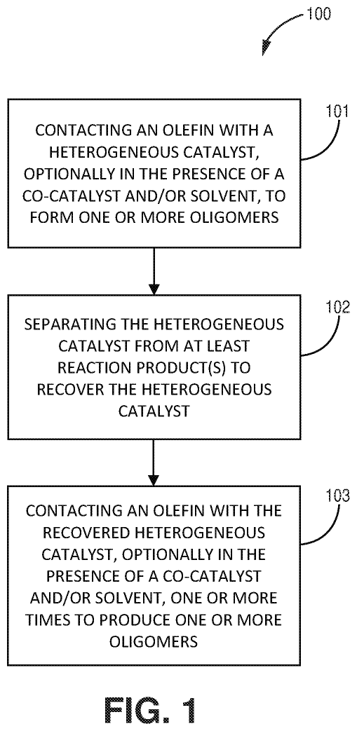

FIG. 1 is a flowchart of a method of oligomerization, according to one or more embodiments of the present disclosure. As shown in FIG. 1, the method 100 comprises contacting 101 one or more olefins with a heterogeneous catalyst, optionally in the presence of one or more co-catalysts and/or one or more solvents, to form one or more oligomers. The heterogeneous catalyst can include any of the metal-organic frameworks of the present disclosure and can optionally include a support. In some embodiments, the heterogeneous catalyst is a solid heterogeneous catalyst. The heterogeneous catalyst can be readily and easily separated from reaction mixtures, reaction products, and/or reagents, and subsequently reused to form one or more additional oligomers. Accordingly, the method 100 can optionally further comprise separating 102 the heterogeneous catalyst from at least reaction product(s) to recover the heterogeneous catalyst and repeating step 101 one or more times with the recovered heterogeneous catalyst (e.g., see step 103 in FIG. 1).

In some embodiments, an olefin (e.g., olefins) or olefin-containing feed stream (e.g., feed stream containing one or more olefins) is contacted with the heterogeneous catalyst. Any olefins capable of being oligomerized in the presence of the heterogeneous catalyst and/or according to the method 100 can be used herein and thus are not particularly limited. Examples of olefins that can be oligomerized according to the methods disclosed herein include, without limitation, ethylene, propylene, butene, pentene, hexene, isomers thereof, and the like. In certain embodiments, the olefins include at least ethylene, and the ethylene undergoes oligomerization to form one or more linear alpha olefins. In certain embodiments, the olefins include at least ethylene, and the ethylene undergoes dimerization to form one or more C₄ hydrocarbons (e.g., C₄ olefins). In certain embodiments, the olefin includes at least propylene, and the propylene undergoes dimerization to form one or more C₆ hydrocarbons. In certain embodiments, the feed stream contains one or more olefins (e.g., ethylene, propylene, etc.). In certain embodiments, the feed stream is a substantially pure feedstock of an olefin. In certain embodiments, the feed stream contains an olefin, such as ethylene, produced from other processes, such as oxidative coupling of methane, metathesis of butenes and methanol to olefins, among others. In some embodiments, more than one olefin is contacted with the heterogeneous catalyst and oligomerized. In some embodiments, more than one olefin is contacted with the heterogeneous catalyst and not oligomerized. In some embodiments, a feed stream containing one or more olefins further comprises additional components. The additional components can include active and/or inert species. In certain embodiments, the feed stream further contains one or more of C₁-C₁₀ hydrocarbons, nitrogen, ammonia, NOₓ, hydrogen, COₓ, and SOₓ, where x is at least 1.

The contacting 101 can proceed by bringing the olefin(s) and heterogeneous catalyst into physical contact, or immediate or close proximity, under oligomerization conditions. In some embodiments, the contacting proceeds by exposing the olefins to the heterogeneous catalyst. In some embodiments, the contacting proceeds by reacting olefins over the heterogeneous catalyst. In some embodiments, the heterogeneous catalyst can be disposed in a reactor or reaction vessel, and the contacting proceeds by introducing a feed stream containing one or more olefins to the reaction vessel. The reactors/reaction vessels are not particularly limited. Examples of suitable reactors include, but are not limited to, tank reactors and tubular reactors which can be configured to operate as batch, semi-batch, or continuously stirred tank reactors, fluidized bed reactors, or fixed bed reactors, or trickle bed reactors. The oligomerization conditions, including temperatures and pressures, are discussed in more detail below.

The heterogeneous catalysts can achieve a turnover frequency in the range of 41,500 moles of monomer consumed per mole of metal per hour to 1,100,000 moles of monomer consumed per mole of metal per hour, or greater depending on the oligomerization conditions under which the contacting is performed, or any increment thereof. In certain embodiments, the turnover frequency of the metal-organic frameworks is at least about 120,000 moles of monomer (e.g., ethylene) consumed per mole of active metal per hour. In some embodiments, the turnover frequency of the metal-organic frameworks is greater than about 500 moles of monomer (e.g., ethylene) consumed per mole of active metal per hour; about 41,500 moles of monomer (e.g., ethylene) consumed per mole of active metal per hour; about 90,000 moles of monomer (e.g., ethylene) consumed per mole of active metal per hour; about 120,000 moles of monomer (e.g., ethylene) consumed per mole of active metal per hour; about 140,000 moles of monomer (e.g., ethylene) consumed per mole of active metal per hour; about 160,000 moles of monomer (e.g., ethylene) consumed per mole of active metal per hour; about 170,000 moles of monomer (e.g., ethylene) consumed per mole of active metal per hour; about 250,000 moles of monomer (e.g., ethylene) consumed per mole of active metal per hour; about 500,000 moles of monomer (e.g., ethylene) consumed per mole of active metal per hour; about 960,000 moles of monomer (e.g., ethylene) consumed per mole of active metal per hour; or about 1,100,000 moles of monomer (e.g., ethylene) consumed per mole of active metal per hour.

The oligomerization can be performed at or over temperatures in the range of about −15° C. to about 500° C. For example, in some embodiments, the oligomerization is performed at a temperature of about 500° C., about 450° C., about 400° C., about 375° C., about 350° C., about 340° C., about 330° C., about 320° C., about 310° C., about 300° C., about 290° C., about 280° C., about 270° C., about 260° C., about 250° C., about 240° C., about 230° C., about 220° C., about 210° C., about 200° C., about 190° C., about 180° C., about 170° C., about 160° C., about 150° C., about 140° C., about 130° C., about 120° C., about 110° C., about 100° C., about 90° C., about 80° C., about 70° C., about 60° C., about 50° C., about 40° C., about 30° C., about 20° C., about 10° C., about 0° C., about −10° C., about −15° C., or any increment between those temperatures. In some embodiments, the oligomerization is performed at a temperature of less than about 60° C., less than about 55° C., less than about 50° C., less than about 45° C., less than about 40° C., less than about 35° C., less than about 30° C., less than about 25° C., less than about 20° C., less than about 15° C., or less than about 10° C. In certain embodiments, the oligomerization is performed at a temperature of about 35° C. In certain embodiments, the oligomerization is performed at a temperature of about 25° C. In certain embodiments, the oligomerization is performed at a temperature in the range of about −15° C. to about 350° C.

The oligomerization can be performed at or over pressures in the range of about 0.1 bar to about 300 bar. In some embodiments, the oligomerization is performed at a pressure of about 75 bar, about 70 bar, about 65 bar, about 60 bar, about 55 bar, about 50 bar, about 45 bar, about 40 bar, about 35 bar, about 30 bar, about 25 bar, about 20 bar, about 15 bar, about 10 bar, about 5 bar, about 1 bar, about 0.9 bar, about 0.8 bar, about 0.7 bar, about 0.6 bar, about 0.5 bar, about 0.4 bar, about 0.3 bar, about 0.2 bar, about 0.1 bar, or any increment between those pressures. In some embodiments, the oligomerization is performed at a pressure of less than about 22 bar and/or greater than about 25 bar. In certain embodiments, the oligomerization is performed at a pressure of about 50 bar. In certain embodiments, the oligomerization is performed at a pressure of about 30 bar. In certain embodiments, the oligomerization is performed at a pressure of about 20 bar. In some embodiments, the oligomerization is performed at a pressure between about 5 and 10 bar. In some embodiments, the oligomerization is performed at a pressure between about 6 to 7 bar (e.g., about 6.8 bar).

The oligomerization can optionally proceed in the presence of one or more co-catalysts. The co-catalysts can include one or more of alkyl aluminum compounds optionally comprising halides, alkyl magnesium compounds optionally comprising halides, aluminoxanes, alkyl lithiums, organoboron compounds, organic compounds capable of acting as proton donors, and the like. Examples of suitable co-catalysts include, but are not limited to, aluminoxanes (e.g., methylaluminoxane, ethylaluminoxane, modified methylaluminoxane), ethylaluminum dichloride, diethylaluminum chloride, ethylaluminum sesquichloride, methylaluminum dichloride, isobutylaluminum dichloride, tributylaluminum, tri-n-octylaluminum, triethylaluminum, trimethylaluminum, ethyl magnesium bromide, and methyllithium. Examples of organoboron compounds include tris(aryl)boranes, such as tris(perfluorophenyl)borane, tris(3,5-bis(trifluoromethyl)phenyl)borane, tris(2,3,4,6-tetrafluorophenyl)borane, tris(perfluoronaphthyl)borane, tris(perfluorobiphenyl)borane, and their derivatives. Examples of organoboron compounds also include (aryl)borates associated with a triphenylcarbenium cation or with a trisubstituted ammonium cation such as triphenylcarbenium tetrakis(perfluorophenyl)borate, N,N-dimethylanilinium tetrakis(perfluorophenyl)borate, N,N-diethylanilinium tetrakis(3,5-bis(trifluoromethyl)phenyl)borate, triphenylcarbenium tetrakis(3,5-bis(trifluoromethyl)phenyl)borate, and the like.

In embodiments including co-catalysts, the amount of co-catalyst used in the method can be in the range of about 0 to about 250,000 equivalents, or any increment thereof. For example, in some embodiments, the amount of co-catalyst is about 1000 equivalents. In some embodiments, the amount of co-catalyst is about 2000 equivalents. In some embodiments, the amount of co-catalyst is about 3000 equivalents. In some embodiments, the amount of co-catalyst is about 4000 equivalents.

In some embodiments, either in addition or in the alternative, the oligomerization can optionally proceed in the presence of one or more solvents. The solvents can include organic solvents, such as ethers, alcohols, chlorinated solvents and the saturated or unsaturated, aromatic or non-aromatic, cyclic or non-cyclic hydrocarbons. Examples of suitable solvents include, but are not limited to, water, methanol, ethanol, propanol, benzene, p-cresol, toluene, xylene, diethyl ether, glycol, diethyl ether, petroleum ether, hexane, cyclohexane, pentane, heptane, butane, isobutene, monoolefins or diolefins comprising between 4 to 20 carbon atoms, methylene chloride, mesitylene, ethylbenzene, dichloromethane, chlorobenzene, chloroform, carbon tetrachloride, dioxane, tetrahydrofuran (THF), dimethyl sulfoxide (DMSO), dimethylformamide, hexamethyl-phosphoric triamide, ethyl acetate, pyridine, triethylamine, picoline, ionic liquids, mixtures thereof, or the like.

The one or more oligomers can include one or more hydrocarbons. The one or more hydrocarbons can have a chain length of four or more carbon atoms. For example, in some embodiments, the olefin includes ethylene and the one or more oligomers includes C₄ hydrocarbons. In some embodiments, the one or more oligomers includes one or more butenes. In some embodiments, the one or more butenes includes one or more of 1-butene, trans-2-butene, cis-2-butene, iso-butene (2-methyl-1-propene), etc. In some embodiments, the feed stream contains mostly ethylene (e.g., at least or more than 50% ethylene) and the reaction products comprise mostly butenes (e.g., at least or more than 50% butenes by weight based on total weight of reaction products). In some embodiments, the butenes include predominantly 1-butene and optionally one or more of trans-2-butene, cis-2-butene, iso-butene, etc. For example, in certain embodiments, the reaction products comprise at least 70% 1-butene by weight (e.g., based on total weight of reaction products), at least about 71% 1-butene, at least about 72% 1-butene, and so on, up to about 100% 1-butene by weight.

In some embodiments, the heterogeneous catalysts can produce one or more butenes (e.g., 1-butene) with a selectivity of at least about 80%. For example, in some embodiments, the heterogeneous catalysts can produce one or more butenes with a selectivity of at least about 80%, at least about 90%, at least about 91%, at least about 91.7%, at least about 91.8%, at least about 92%, at least about 93%, at least about 94%, at least about 95%, at least about 96%, at least about 97%, at least about 97.4%, at least about 98%, at least about 99%, at least about 99.99%, or greater, or any increment thereof. In some embodiments, the metal-organic frameworks can be utilized as catalysts to produce 1-butene with a selectivity of at least about 85%, at least about 85.5%, at least about 86%, at least about 86.5%, at least about 87%, at least about 87.5%, at least about 87.6%, at least about 88%, at least about 89%, at least about 89.5%, at least about 89.6%, at least about 90%, at least about 91.5%, at least about 92%, at least about 92.5%, at least about 93%, at least about 93.5%, at least about 94%, at least about 94.5%, at least about 94.6%, or greater, or any increment thereof. In other embodiments, the heterogeneous catalysts can produce one or more butenes with a selectivity of at least about 60%. The one or more butenes can include one or more of any of the butenes disclosed herein.

In some embodiments, the olefin includes ethylene and the one or more oligomers includes one or more linear alpha olefins. The one or more linear alpha olefins can have chain-lengths ranging from 4 to 30-plus carbon atoms. For example, in some embodiments, the one or more linear alpha olefins includes one or more of C₄ linear alpha olefins, C₆ linear alpha olefins, C₈ linear alpha olefins, C₁₀ linear alpha olefins, C₁₂ linear alpha olefins, C₁₄ linear alpha olefins, C₁₆ linear alpha olefins, C₁₈ linear alpha olefins, C₂₀ linear alpha olefins, C₂₂ linear alpha olefins, C₂₂ linear alpha olefins, C₂₄ linear alpha olefins, C₂₆ linear alpha olefins, C₂₈ linear alpha olefins, C₃₀ linear alpha olefins, and C₃₀₊ linear alpha olefins. In some embodiments, the one or more oligomers include C₄-C₂₀₊ linear alpha olefins. In some embodiments, the one or more oligomers include C₄-C₃₀₊ linear alpha olefins. In some embodiments, the one or more oligomers include C₄-C₈ linear alpha olefins. In some embodiments, the one or more oligomers include C₆-C₁₀ linear alpha olefins. In some embodiments, the one or more oligomers include C₁₂-C₁₈ linear alpha olefins. In some embodiments, the one or more oligomers include C₂₀-C₂₄ linear alpha olefins. In some embodiments, the one or more oligomers include C₂₄-C₃₀ linear alpha olefins. In some embodiments, the one or more oligomers include C₂₀-C₃₀ linear alpha olefins. In some embodiments, the one or more linear alpha olefins includes one or more of C₄-C₈ linear alpha olefins, C₆-C₈ linear alpha olefins, C₆-C₁₀ linear alpha olefins, C₁₀-C₁₂ linear alpha olefins, C₁₀-C₁₆ linear alpha olefins, C₁₂-C₁₈ linear alpha olefins, C₁₆-C₁₈ linear alpha olefins, C₂₀-C₂₄ linear alpha olefins, C₂₄-C₃₀ linear alpha olefins, C₂₀-C₃₀ linear alpha olefins, and C₂₀-C₃₀₊ linear alpha olefins. In some embodiments, the one or more linear alpha olefins includes one or more of 1-butene, 1-pentene, 1-hexene, 1-octene, 1-decene, 1-dodecene, 1-tetradecene, 1-hexadecene, 1-octadecene, 1-eicosene, 1-docosene, 1-tetracosene, 1-hexacosene, 1-octacosene, and 1-triacontene.

In some embodiments, one or more linear alpha olefins is produced with a selectivity of at least about 60%. For example, in some embodiments, the C₄-C₂₀₊ linear alpha olefins are produced with a selectivity of at least about 60%. In other embodiments, one or more linear alpha olefins is produced with a selectivity in the range of about 0% to about 100%, or any incremental value or subrange between that range. In some embodiments, a product distribution of the one or more linear alpha olefins includes about 5-15 wt. % C₄ linear alpha olefin, about 20-65 wt. % C₆-C₁₀ linear alpha olefins, about 20-65 wt. % C₁₂-C₁₈ linear alpha olefins, and/or about 0.1-10 wt. % C₂₀₊ linear alpha olefins. In some embodiments, the product distribution of the one or more linear alpha olefins includes about 11 wt. % C₄ linear alpha olefin. In some embodiments, the product distribution of the one or more linear alpha olefins includes about 42 wt. % C₆-C₁₀ linear alpha olefins. In some embodiments, the product distribution of the one or more linear alpha olefins includes about 45 wt. % C₁₂-C₁₈ linear alpha olefins. In some embodiments, the product distribution of the one or more linear alpha olefins includes about 2 wt. % C₂₀₊ linear alpha olefins. In some embodiments, a rate of production of one or more linear alpha olefins is at least about 100 grams per gram of catalyst per hour. For example, in some embodiments, a rate of production of C₄-C₂₀₊ linear alpha olefins is at least about 100 grams per gram of catalyst per hour. In some embodiments, a rate of production of the one or more linear alpha olefins is about 5 grams to about 1000 grams per gram of catalyst per hour. The one or more linear alpha olefins can include any combination of the linear alpha olefins.

In some embodiments, the olefin includes propylene and the one or more oligomers includes one or more C₆ hydrocarbons (e.g., C₆ dimers). For example, in some embodiments, the one or more oligomers includes one or more C₆ dimers. In some embodiments, the one or more C₆ dimers includes one or more of 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2,3-dimethyl-2-butene, 3,3-dimethyl-1-butene, methylcyclopentene, cyclohexene, and the like. In some embodiments, the one or more C₆ dimers include one or more of methyl-pentenes, dimethyl-butenes, and n-hexenes. In some embodiments, the one or more C₆ dimers includes 4-methyl-1-pentene. In some embodiments, the one or more C₆ dimers includes 2-methyl-1-pentene. In some embodiments, the one or more C₆ dimers includes 2,3-dimethylbutene. In some embodiments, the one or more C₆ dimers includes 2,3-dimethyl-1-butene and/or 2,3-dimethyl-2-butene.

In some embodiments, the one or more C₆ hydrocarbons is produced with a selectivity of at least about 90%. For example, in some embodiments, one or more C₆ dimers are produced with a selectivity of at least about 90%. In other embodiments, the one or more C₆ dimers is produced with a selectivity in the range of about 0% to about 100%, or any incremental value or subrange between that range. In some embodiments, the product distribution includes at least 60 wt. % C₆ dimers. For example, in some embodiments, the product distribution includes about 97 wt. % C₆ dimers. In other embodiments, the production distribution of one or more C₆ dimers is in the range of about 0 wt. % to about 100 wt. %, or any incremental value or subrange between that range. In some embodiments, a rate of production of the one or more C₆ hydrocarbons is about 2700 grams per gram of catalyst per hour. In some embodiments, a rate of production of the one or more C₆ dimers is about 2700 grams per gram of catalyst per hour. In other embodiments, a rate of production of the one or more C₆ dimers is about 100 grams to about 3500 grams per gram of catalyst per hour.

At step 102, the metal-organic framework can be separated from at least the reaction products and optionally the reaction solution (e.g., co-catalysts, solvents, byproducts, unreacted reagents, residual products, reaction products, etc.). In some embodiments, the metal-organic framework is a heterogeneous catalysts that can be easily separated and recovered from reagents, products, and other chemical species. For example, in certain embodiments, the metal-organic framework is separated from one or more of reaction products, C1-C10 hydrocarbons, nitrogen, ammonia, NOₓ, hydrogen, COₓ, and SOₓ, where x is at least 1. In certain embodiments, the metal-organic framework is a solid heterogeneous catalyst and thus can be easily separated from liquids, gas/vapor mixtures, or combinations thereof. At least one advantage of the metal-organic frameworks is that they can be recovered and reused in additional reaction cycles. Accordingly, once recovered, step 101 (and step 102) can be repeated one or more times with recovered metal-organic frameworks.

In some embodiments, a method of oligomerization may include contacting ethylene with a heterogeneous catalyst to form one or more butenes, wherein the heterogeneous catalyst comprises a metal-organic framework and an optional support, the metal-organic framework comprising a plurality of first metal ions coordinated to one or more ligands, wherein each of the one or more ligands has only one N-heterocyclic aromatic group.

In some embodiments, a method of oligomerization may include contacting ethylene with a heterogeneous catalyst to form one or more linear alpha olefins, wherein the heterogeneous catalyst comprises a metal-organic framework and an optional support, the metal-organic framework comprising a plurality of first metal ions coordinated to one or more ligands, wherein each of the one or more ligands has only one N-heterocyclic aromatic group.

In some embodiments, a method of oligomerization may include contacting propylene with a heterogeneous catalyst to form one or more C₆ dimers, wherein the heterogeneous catalyst comprises a metal-organic framework and an optional support, the metal-organic framework comprising a plurality of first metal ions coordinated to one or more ligands, wherein each of the one or more ligands has only one N-heterocyclic aromatic group.

In some embodiments, a method of producing C₆ products from propylene includes propylene reacting over a heterogenous catalyst that is composed of a metal-doped (e.g. Ti, Ni, Cu, Co, Cr and V) metal-organic framework (MOF) wherein each ligand comprises only one N-heterocyclic aromatic group (ligand e.g. imidazolate, triazolate and tetrazolate). Once reacted with propylene, it is capable of producing C₆ products with a selectivity of at least 90% and at a rate of production of at least 2700 g of products per gram of catalyst per hour.

In some embodiments, a method of producing a wide range of linear alpha olefins (C₄-C₂₀₊) from ethylene includes reacting ethylene over a heterogenous catalyst that is composed of a metal-doped (e.g. Ti, Ni, Cu, Co, Cr and V) metal-organic framework (MOF) wherein each ligand comprises only one N-heterocyclic aromatic group (ligand e.g. imidazolate, triazolate and tetrazolate). Once reacted with ethylene, it is capable of producing wide range of linear alpha olefins (C₄-C₂₀,) with a selectivity of at least 60% and at a rate of production of at least 100 g of products per gram of catalyst per hour.

In some embodiments, a method of oligomerization comprises contacting at least ethylene with a heterogeneous oligomerization catalyst to produce hydrocarbons, wherein the hydrocarbons include one or more linear alpha olefins and wherein the heterogeneous oligomerization catalyst includes a metal-organic framework comprising a first metal ion component coordinated to a ligand having only one N-heterocyclic aromatic group.

In some embodiments, a method of oligomerization comprises contacting propylene with a heterogeneous oligomerization catalyst to produce one or more hydrocarbons having six carbon atoms, wherein the heterogeneous oligomerization catalyst is a metal-organic framework comprising a first metal component coordinated to a ligand having only one N-heterocyclic aromatic group.

In some embodiments, a method of oligomerization comprises contacting a monomer with a heterogeneous catalyst to form an oligomer, wherein the heterogeneous catalyst comprises a metal-organic framework, the metal-organic framework comprising a plurality of first metal ions coordinated to ligands having only one N-heterocyclic aromatic group, and optionally a metal dopant, wherein the metal dopant includes a plurality of second metal ions associated with the metal-organic framework.

In certain embodiments, the monomer is ethylene and the oligomer is 1-butene, wherein the 1-butene is produced by ethylene dimerization. In certain embodiments, the oligomer is produced with a selectivity of at least about 90%. In certain embodiments, the turnover frequency of the heterogeneous catalyst is at least about 120,000 moles of monomer consumed per mole of active metal per hour.

In certain embodiments, the first metal ion comprises a metal selected from the group consisting of Zn, Ti, Ni, Cu, Cr, V, Cd, or Ru. In certain embodiments, the first metal ion content is in the range of about 1% to about 60% by weight of the heterogeneous catalyst. In certain embodiments, the metal-organic framework further comprises a metal dopant, wherein the metal dopant comprises a plurality of second metal ions including a metal selected from the group consisting of Ti, Ni, Cu, Cr, or V. In certain embodiments, the metal dopant content is in the range of about 0% to about 60% by weight of the catalyst. In certain embodiments, the N-heterocyclic aromatic group is selected from the group consisting of imidazolates, triazolates, or tetrazolates. In certain embodiments, the first metal ions and/or second metal ions are coordinated to the ligand through either a nitrogen heteroatom of the N-heterocyclic aromatic group, or a coordinating O- or S-donor moiety of the ligand.

In certain embodiments, a bite angle of the heterogeneous catalyst is about 109°. In certain embodiments, the heterogeneous catalyst has a particle size in the range of 5 nm to about 5000000 nm.

In certain embodiments, the monomer contacts the heterogeneous catalyst at a temperature in the range of −15° C. to 350° C. In certain embodiments, the monomer contacts the heterogeneous catalyst at a pressure in the range of 0.1 bar to 75 bars. In certain embodiments, the feed stream further comprises one or more of C₁ to C₁₀ hydrocarbons, nitrogen, ammonia, NOₓ, hydrogen, COₓ, and SOₓ, where x is at least 1. In certain embodiments, the butene includes at least 1-butene and optionally isomers of butenes selected from trans-2-butene, cis-2-butene, isobutene, or combinations thereof. In certain embodiments, the feed stream, products, or both further comprise C1-C10 hydrocarbons, inert species such as N₂, ammonia, NOx, hydrogen, COₓ, SOₓ, or combinations thereof.

In some embodiments, a heterogeneous catalyst comprises a metal-organic framework, the metal-organic framework comprising a plurality of first metal ions, each of the first metal ions coordinated to a ligand having only one N-heterocyclic aromatic group, and optionally a metal dopant, wherein the metal dopant includes a plurality of second metal ions associated with the metal-organic framework. The heterogeneous catalyst can have one or more of the characteristics of provided above and elsewhere throughout the present disclosure.

According to some embodiments, a method of oligomerization may include contacting an olefin with a heterogeneous catalyst to form one or more oligomers, wherein the heterogeneous catalyst comprises a metal-organic framework and an optional support, the metal-organic framework comprising a plurality of first metal ions coordinated to one or more ligands, wherein each of the one or more ligands has only one N-heterocyclic aromatic group.

In some embodiments, the olefin includes ethylene and the one or more oligomers includes one or more butenes. In some embodiments, the one or more butenes includes one or more of 1-butene, trans-2-butene, cis-2-butene, and isobutene. In some embodiments, the one or more butenes includes 1-butene. In some embodiments, the one or more butenes is produced with a selectivity of at least about 80%. In some embodiments, the turnover frequency of the heterogeneous catalyst is at least about 120,000 moles of ethylene consumed per mole of active metal per hour.

In some embodiments, the olefin includes ethylene and the one or more oligomers includes one or more linear alpha olefins. In some embodiments, the one or more linear alpha olefins includes one or more of 1-butene, 1-pentene, 1-hexene, 1-octene, 1-decene, 1-dodecene, 1-tetradecene, 1-hexadecene, 1-octadecene, 1-eicosene, 1-docosene, 1-tetracosene, 1-hexacosene, 1-octacosene, and 1-triacontene. In some embodiments, the one or more linear alpha olefins includes one or more of C₄-C₈ linear alpha olefins, C₆-C₈ linear alpha olefins, C₆-C₁₀ linear alpha olefins, C₁₀-C₁₂ linear alpha olefins, C₁₀-C₁₆ linear alpha olefins, C₁₆-C₁₈ linear alpha olefins, and C₂₀-C₃₀₊ linear alpha olefins. In some embodiments, the one or more linear alpha olefins is produced with a selectivity of at least about 60%. In some embodiments, a rate of production of the one or more linear alpha olefins is at least about 100 grams per gram of catalyst per hour.

In some embodiments, the olefin includes propylene and the one or more oligomers includes one or more C₆ dimers. In some embodiments, the one or more C₆ dimers includes one or more of 1-hexene, 2-hexene, 3-hexene, 2-methyl-1-pentene, 2-methyl-2-pentene, 3-methyl-1-pentene, 3-methyl-2-pentene, 4-methyl-1-pentene, 4-methyl-2-pentene, 2,3-dimethyl-1-butene, 2,3-dimethyl-2-butene, 3,3-dimethyl-1-butene, methylcyclopentene, and cyclohexene. In some embodiments, the one or more C₆ dimers includes 4-methyl-1-pentene. In some embodiments, the one or more C₆ dimers includes 2-methyl-1-pentene. In some embodiments, the one or more C₆ dimers includes at least 2,3-dimethylbutene. In some embodiments, the one or more C₆ dimers is produced with a selectivity of at least about 90%. In some embodiments, a rate of production of the one or more C₆ dimers is at least about 2700 grams per gram of catalyst per hour.

In some embodiments, the plurality of first metal ions includes one of Zn, Ti, Ni, Cu, Cr, V, Cd, Co, Zr, Fe, Al, Mn, and Ru. In some embodiments, the plurality of first metal ions includes one of Ti, Ni, Cu, Co, Cr, and V. In some embodiments, the plurality of first metal ions includes one of Co, Ni, and Zn. In some embodiments, the plurality of first metal ions are coordinated to each ligand through a nitrogen heteroatom of the N-heterocyclic aromatic group, or a coordinating O- or S-donor moiety of the ligand. In some embodiments, the first metal ion content is in the range of about 0.01% to about 60% by weight of the heterogeneous catalyst.

In some embodiments, the one or more ligands includes one of pyrrolate, pyrazolate, triazolate, imidazolate, oxazolate, tetrazolate, pyridinate, thiazolate, oxadiazolate, purinate, quinolonate, and indolate. In some embodiments, the one or more ligands includes one of imidazolate, triazolate, and tetrazolate. In some embodiments, the one or more ligands has the formula: R^{a}—(N-heterocyclic aromatic group)-R^{b}, wherein R^{a} and R^{b} are the same or different and wherein each of R^{a} and R^{b} is independently —H, -alkyl, —NO₂, —R′, —F, —Cl, —Br, —I, —CN, —NC, —SO₃R′, —SO₃H, —OR′, —OH, —SR′, —SH, —PO₃R′, —PO₃H, —CF₃, —NR′2, —NHR′, and —NH₂, wherein each R′ is the same or different and wherein each R′ is optionally substituted alkyl or optionally substituted aryl, provided that R′ is not an N-heterocyclic aryl.

In some embodiments, the heterogeneous catalyst further includes a metal dopant. In some embodiments, the metal dopant includes a plurality of second metal ions and wherein the plurality of second metal ions includes one of Ti, Ni, Cu, Cr, Co, Zr, Fe, Al, Mn, Ru, Pd, Pt, and V. In some embodiments, the plurality of second metal ions are coordinated to each ligand through a nitrogen heteroatom of the N-heterocyclic aromatic group, or a coordinating O- or S-donor moiety of the ligand. In some embodiments, the second metal ion content is in the range of about 0% to about 60% by weight of the heterogeneous catalyst.

In some embodiments, the heterogeneous catalyst further includes a co-catalyst. In some embodiments, wherein the co-catalyst includes one or more of methylaluminoxane, ethylaluminoxane, modified methylaluminoxane, ethylaluminum dichloride, diethylaluminum chloride, ethylaluminum sesquichloride, methylaluminum dichloride, isobutylaluminum dichloride, tributylaluminum, tri-n-octylaluminum, triethylaluminum, trimethylaluminum, ethyl magnesium bromide, methyllithium, tris(perfluorophenyl)borane, tris(3,5-bis(trifluoromethyl)phenyl)borane, tris(2,3,4,6-tetrafluorophenyl)borane, tris(perfluoronaphthyl)borane, tris(perfluorobiphenyl)borane, triphenylcarbenium tetrakis(perfluorophenyl)borate, N,N-dimethylanilinium tetrakis(perfluorophenyl)borate, N,N-diethylanilinium tetrakis(3,5-bis(trifluoromethyl)phenyl)borate, and triphenylcarbenium tetrakis(3,5-bis(trifluoromethyl)phenyl)borate. In some embodiments, the co-catalyst is methyl aluminoxane (MAO).

In some embodiments, the heterogeneous catalyst has a particle size in the range of about 5 nm to about 5000000 nm. In some embodiments, the olefin and the heterogeneous catalyst are contacted at a temperature in the range of −15° C. to 500° C. In some embodiments, the olefin and the heterogeneous catalyst are contacted at a pressure in the range of about 0.1 bar to about 300 bar. In some embodiments, a bite angle of the heterogeneous catalyst is in the range of about 50° to about 180°. In some embodiments, the olefin is included in a feed stream and wherein the feed stream further includes one or more of C₁-C₁₀ hydrocarbons, nitrogen, ammonia, NOR, hydrogen, COₓ, and SOₓ, where x is at least 1.

The following Examples are intended to illustrate the above invention and should not be construed as to narrow its scope. One skilled in the art will readily recognize that the Examiners suggest many other ways in which the invention could be practiced. It should be understood that numerous variations and modifications may be made while remaining within the scope of the invention.

### Example 1

### Preparation of Catalysts

1-Butene is used as a monomer added to ethylene to produce a very valuable plastic material ‘polyethylene’ that accounts for more than 34% of the plastics market. Currently, 1-Butene is produced via several processes as a byproduct from the refinery/steam cracking or ethylene oligomerization and as the main product from ethylene dimerization.

Currently, the commercialized technology for direct production of 1-butene from ethylene is Alphabutol Technology (Axens in cooperation with SABIC) with 30 licenses around the world with a total production of 708,000 MTPY of 1-butene (25% of worldwide production) and has been in the market for 33 years. The technology uses a homogenous catalyst (where products, catalysts, and co-catalysts are in one phase) and thus needs to be separated after the reaction in order to get the products. This is a very difficult process which consumes huge amounts of energy and money. Moreover, the catalyst is reported to have very poor stability, where a loss of 50% in activity happens in a single hour. After separation, the catalyst must be treated with chemicals to remove harmful materials and the treated catalyst cannot be used again to produce products, therefore, it has to be disposed of immediately. Homogenous catalysts have been reported to have very high selectivity and activity for dimerization of olefins. Among examples reported is a homogenous catalyst composition consisting of at least a nickel precursor with at least one ligand of the imino-imidazole type that is capable of converting ethylene to butenes as mentioned in patent application U.S. Pat. No. 9,545,623. However, this catalyst system is in liquid phase that is totally different from the solid heterogeneous catalyst used in this invention. Heterogeneous catalyst systems are environmentally friendly options to overcome homogenous catalysts problems. Among examples using this category of catalysts is Metal-Organic Frameworks (MOFs) where changing ligand properties gives totally different functionalities. One type mentioned in U.S. Pat No. 2018/0250664 is to use MOFs in the conversion of ethylene to 1-butene that comprises of at least one ligand wherein each ligand comprises at least two unsaturated N-heterocyclic aromatic groups. It is different from the MOF disclosed herein where each ligand consists of one and only one N-heterocyclic aromatic group that can efficiently give different properties, catalytic activity, and selectivity. Moreover, among significant differences are bite angle and steric environment which play critical roles in the catalysis.

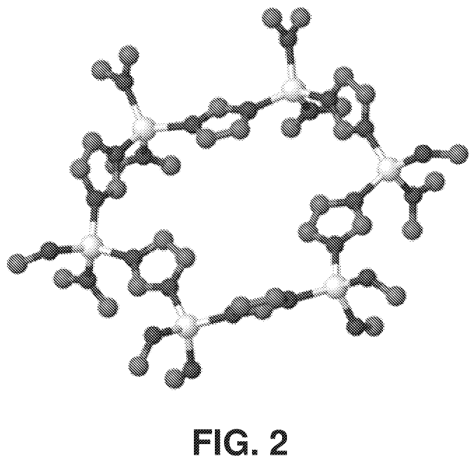

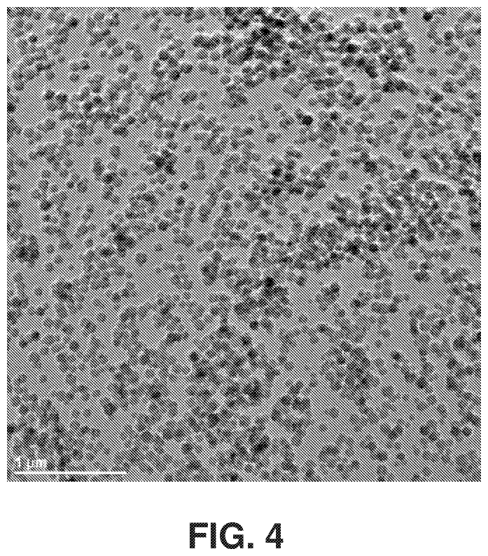

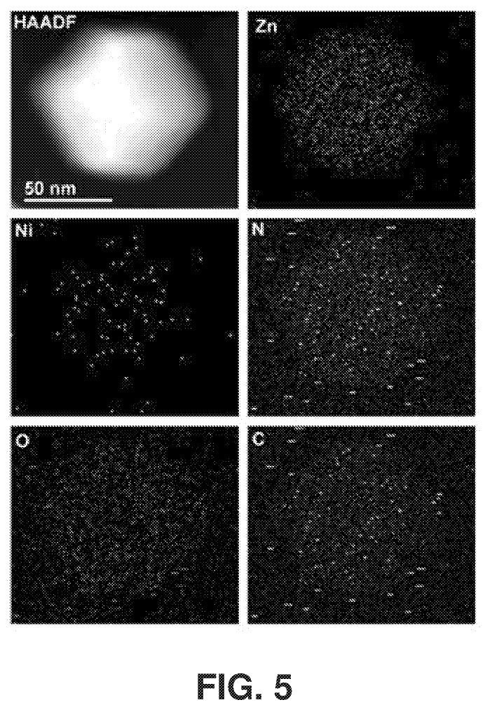

Accordingly, the following Example relates to various metal doped-metal-organic frameworks (MOFs) comprising metals, metal dopants, and ligands having only one N-heterocyclic group. The metal doped-MOFs included the following MOFs: KAUST-1/4M, KAUST-1/2M, and KAUST-1M. Each was utilized as a solid heterogeneous catalyst for ethylene dimerization using ligands with only one N-heterocyclic group (imidazole). In general, the preparation of the metal-doped MOFs included dissolving a mixture of Zn(NO₃)₂.6H₂O and Ni(NO₃)₂.6H₂O in methanol (solution 1). Another solution (solution 2) comprising methanol and 2-methylimidazole (FIG. 2) was mixed with solution 1 under stirring at about room temperature. The mixed solution was allowed to react for a while, and then was centrifuged and washed with methanol several times. The modeled structure, PXRD pattern, Transmission Electron Microscopy image, Energy Dispersive X-ray Spectroscopy images are shown in FIGS. 2-5, respectively.

### Example 2

### Catalytic Performance of KAUST-1/4 M

The following Example illustrates the catalytic performance of a heterogeneous catalyst (KAUST-1/4M) in the dimerization of ethylene to produce butenes. The heterogeneous catalyst comprised a metal-doped metal-organic framework, the metal-doped metal-organic framework including a ligand having only one N-heterocyclic aromatic group as shown in FIGS. 2-5.

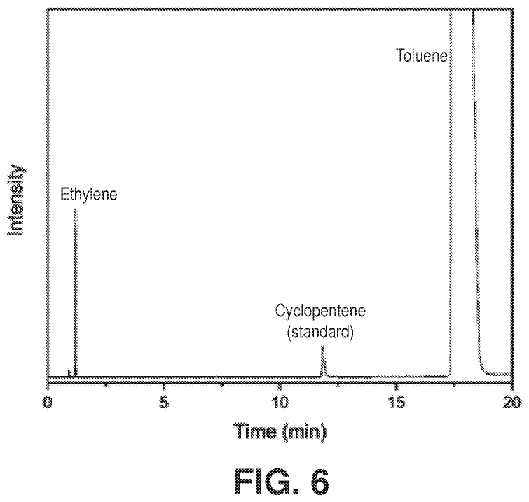

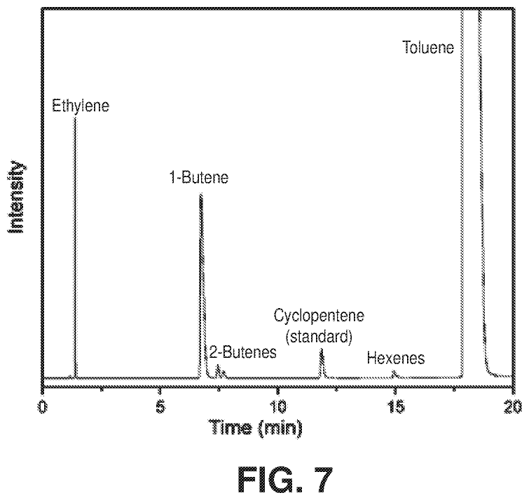

In a typical run, toluene, the catalyst, and a co-catalyst (methyl aluminoxane (MAO)) were added to a tank reactor kept at about 35° C. and stirred. The amount of co-catalyst added was about 2000 equivalents of MAO. The ethylene was fed to the reactor with a total pressure of about 30 bar and kept there for about 10 minutes. Then, the reactor was quenched immediately in a mixture of dry ice and acetone in order to condense all products at a temperature of about −40° C. Thereafter, cyclo-pentene was added as an external standard in order to analyze results in Gas Chromatography (As shown in FIG. 6 (before reaction) and FIG. 7 (after reaction)). KAUST-1/4M catalyst showed a remarkable activity of about 250,000 moles of ethylene consumed per moles of metal per hour (See Table 1 for comparison) with a very good selectivity towards butenes comprising about 95% of products while the other approx. 5% included hexenes. Analyzing butenes product stream, about 89% of the butenes products were 1-butenes while the remainder comprising a mixture of 2-butenes and iso-butene.

### Example 3

### Catalytic Performance of KAUST-1/2 M

The following Example illustrates the catalytic performance of a heterogeneous catalyst (KAUST-1/2 M) in the dimerization of ethylene to produce butenes. The heterogeneous catalyst comprised a metal-doped metal-organic framework, the metal-doped metal-organic framework including a ligand having only one N-heterocyclic aromatic group as shown in FIGS. 2-5.

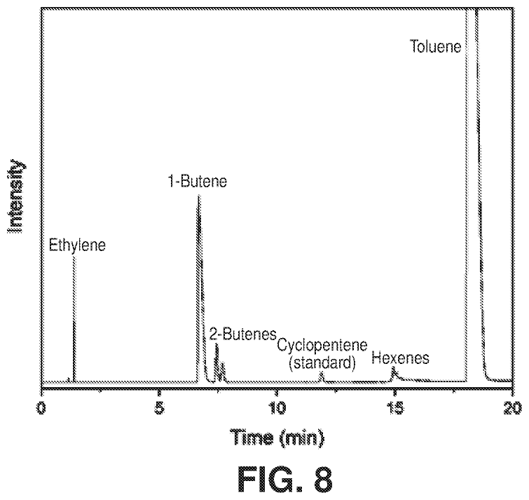

In a typical run, dried toluene, the catalyst, and a co-catalyst (methyl aluminoxane (MAO)) were added and stirred in a tank reactor that was kept at about 35° C. The amount of co-catalyst added was about 4000 equivalents of MAO. The ethylene was fed to the reactor with a total pressure of about 30 bar and kept there for about 10 minutes. Then, the reactor was quenched immediately in a mixture of dry ice and acetone in order to condense all products at a temperature of about −40° C. Thereafter, cyclo-pentene was added as an external standard in order to analyze results in Gas Chromatography (As shown in FIG. 6 (before reaction) and FIG. 8 (after reaction)). KAUST-1/2M catalyst showed a superior activity of about 500,000 moles of ethylene consumed per moles of metal per hour (Table 1 for comparison) with a very good selectivity towards butenes comprising about 94% of products while the other approx. 6% comprised hexenes. Analyzing butenes product stream, about 87.5% of the butenes products are 1-butenes while the rest comprised a mixture of 2-butenes and iso-butene.

### Example 4

### Heterogeneity of Catalytic System

The following Example illustrates the heterogeneity of the catalytic system. The catalytic performance of a heterogeneous catalyst (KAUST-1/4 M) in the dimerization of ethylene to produce butenes, wherein the catalyst comprised a metal-doped metal-organic framework, wherein each ligand comprised only one N-heterocyclic aromatic group as shown in FIGS. 2-5.

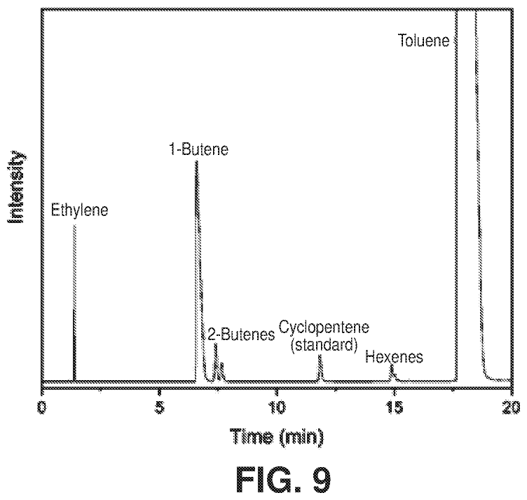

Prior to the run, the heterogeneous catalyst (KAUST-1/4 M) was washed with toluene twice (KAUST-1/4M-Toluene washed). In a typical run, dried toluene, the washed catalyst and a co-catalyst (methyl aluminoxane (MAO)) were added and stirred in a tank reactor that was kept at about 35° C. The amount of co-catalyst added was about 2000 equivalents of MAO. The ethylene was fed to the reactor with a total pressure of about 30 bar and kept there for about 10 minutes. Then, the reactor was quenched immediately in a mixture of dry ice and acetone in order to condense all products at a temperature of about −40° C. After that, cyclo-pentene was added as an external standard in order to analyze results in Gas Chromatography (As shown in FIG. 6 (before reaction) and FIG. 9 (after reaction)). KAUST-1/4M-toluene washed catalyst showed similar activity to that seen in Example 2. An activity of about 250,000 moles of ethylene consumed per moles of metal per hour (Table 1 for comparison) with a very good selectivity towards butenes comprising about 95% of products while the other approx. 5% comprised hexenes. Analyzing the butenes product stream, about 89% of the butenes products were 1-butenes while the rest comprised a mixture of 2-butenes and iso-butene.

### Example 5

### Catalyst Reuse

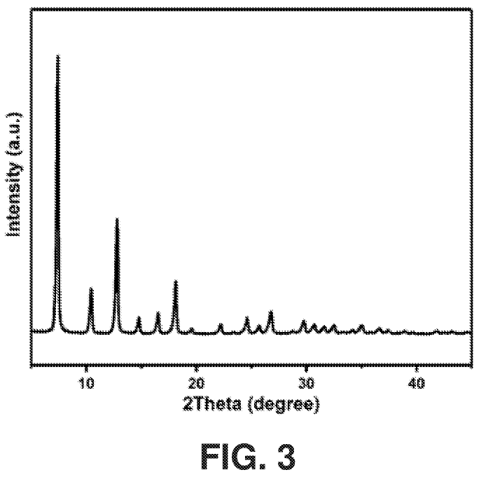

The following Example illustrates that the catalytic system can be reused based on the catalytic performance of a heterogeneous catalyst (KAUST-1/4 M) in the dimerization of ethylene to produce butenes, wherein the catalyst comprised a metal-doped metal-organic framework, wherein each ligand comprised only one N-heterocyclic aromatic group as shown in FIGS. 2-3.

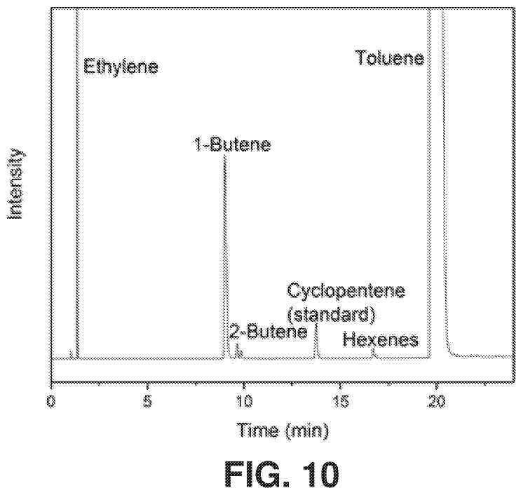

Prior to the run, the heterogeneous catalyst (KAUST-1/4 M) used in Example 2 was collected after reaction and dried at about 80° C. (KAUST-1/4 M-reused). In a typical run, dried toluene, the used catalyst, and a co-catalyst (methyl aluminoxane (MAO)) were added and stirred in a tank reactor that was kept at about 35° C. The amount of co-catalyst added was about 2000 equivalents of MAO. The ethylene was fed to the reactor with a total pressure of about 30 bar and kept there for about 10 minutes. Then, the reactor was quenched immediately in a mixture of dry ice and acetone in order to condense all products at a temperature of about −40° C. After that, cyclo-pentane was added as an external standard in order to analyze results in Gas Chromatography (As shown in FIGS. 6, 10). KAUST-1/4M catalyst showed good activity of about 170,000 moles of ethylene consumed per moles of metal per hour (table 1 for comparison) with a very good selectivity towards butenes comprising about 96% of products while the other approx. 4% comprised hexenes. Analyzing the butenes product stream, about 90% of the butenes products were 1-butenes while the rest comprised a mixture of 2-butenes and iso-butene.

### Example 6

### Effect of Temperature on Catalytic Performance of KAUST-1/4 M

The following Example illustrates the effect of temperature on the catalytic performance of a heterogeneous catalyst (KAUST-1/4 M) comprising a metal-doped metal-organic framework, where each ligand comprised only one N-heterocyclic aromatic group as shown in FIG. 2 and FIG. 3 in the dimerization of ethylene to produce butenes.

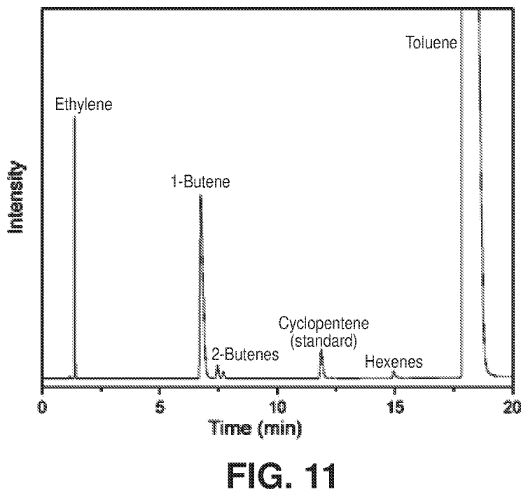

In a typical run, dried toluene, the catalyst and a co-catalyst (methyl aluminoxane (MAO)) were all added and stirred in a tank reactor that is kept at about 25° C. The amount of co-catalyst added was about 2000 equivalents of MAO. The ethylene was fed to the reactor with a total pressure of about 30 bar and kept there for about 10 minutes. Then, the reactor was quenched immediately in a mixture of dry ice and acetone in order to condense all products at a temperature of about −40° C. After that, cyclo-pentene was added as an external standard in order to analyze results in Gas Chromatography (As shown in FIG. 6 (before reaction) and FIG. 11 (after reaction)). KAUST-1/4M catalyst showed an activity of about 140,000 moles of ethylene consumed per moles of metal per hour which was less than when the reactor temperature was about 35° C. (Table 1 for comparison). The catalyst operated at a temperature of about 25° C. gave a very good selectivity towards butenes comprising about 97% of products while the other approx. 3% comprised hexenes. Analyzing the butenes product stream, about 92% of the butenes products were 1-butenes while the rest comprised a mixture of 2-butenes and iso-butene. As shown, the temperature played a critical role in the reaction.

### Example 7

### Effect of Pressure on Catalytic Performance of KAUST-1/4 M

The following Example illustrates the effect of pressure on the catalytic performance of a heterogeneous catalyst (KAUST-1/4 M) comprising a metal-doped metal-organic framework, where each ligand comprised only one N-heterocyclic aromatic group as shown in FIG. 2 and FIG. 3 in the dimerization of ethylene to produce butenes.

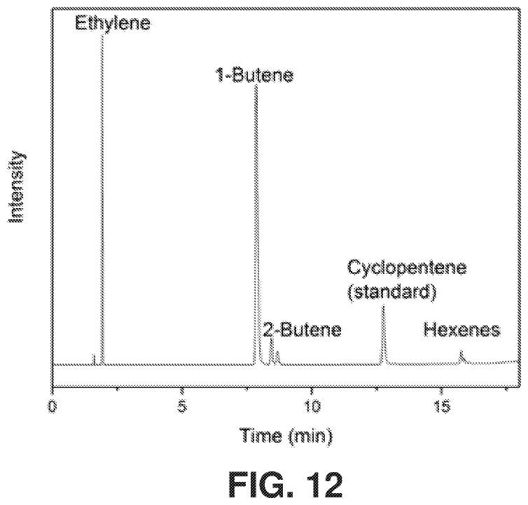

In a typical run, dried toluene, the catalyst and a co-catalyst (methyl aluminoxane (MAO)) were all added and stirred in a tank reactor that was kept at about 35° C. The amount of co-catalyst added was about 2000 equivalents of MAO. The ethylene was fed to the reactor with a total pressure of about 20 bar and kept there for about 10 minutes. Then, the reactor was quenched immediately in a mixture of dry ice and acetone in order to condense all products at a temperature of about −40° C. After that, cyclo-pentene was added as an external standard in order to analyze results in Gas Chromatography (As shown in FIG. 6 (before reaction) and FIG. 12 (after reaction)). KAUST-1/4 M catalyst showed an activity of about 160,000 moles of ethylene consumed per moles of metal per hour (that was less than when the reactor was operated at higher pressure (Table 1 for comparison)) with a very good selectivity towards butenes comprising about 94% of products while the other approx. 3% comprised hexenes. Analyzing the butenes product stream, about 90% of the butenes products were 1-butenes while the rest comprised a mixture of 2-butenes and iso-butene. The pressure therefore affected the reaction.

### Example 8

### Catalytic Performance of KAUST-1 M

The following Example illustrates the catalytic performance of a heterogeneous catalyst (KAUST-1M) comprising a metal-doped metal-organic framework, where each ligand comprised only one N-heterocyclic aromatic group as shown in FIG. 2 and FIG. 3 in the dimerization of ethylene to produce butenes.

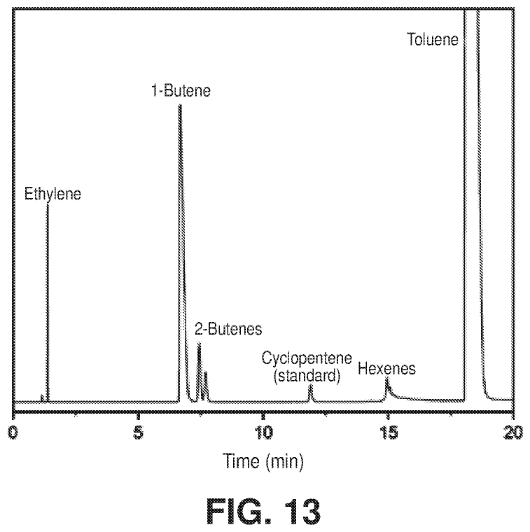

In a typical run, dried toluene, the catalyst and a co-catalyst (methyl aluminoxane (MAO)) were all added and stirred in a tank reactor that was kept at about 35° C. The amount of co-catalyst added was about 4000 equivalents of MAO. The ethylene was fed to the reactor with a total pressure of about 50 bar and kept there for about 10 minutes. Then, the reactor was quenched immediately in a mixture of dry ice and acetone in order to condense all products at a temperature of about −40° C. After that, cyclo-pentene was added as an external standard in order to analyze results in Gas Chromatography (As shown in FIG. 6 and FIG. 13). KAUST-1M catalyst showed a remarkable activity of about 1,100,000 moles of ethylene consumed per moles of metal per hour (Table 1 for comparison) with a very good selectivity towards butenes comprising about 96% of products while the other approx. 4% comprised hexenes. Analyzing the butenes product stream, about 88% of the butenes products were 1-butenes while the rest comprised a mixture of 2-butenes and iso-butene.

### Example 9

### Effect of Co-Catalyst on Catalytic Performance of KAUST-1/4 M

The following Example illustrates the effect of co-catalyst amount on the catalytic performance of a heterogeneous catalyst (KAUST-1/4 M) comprising a metal-doped metal-organic framework, where each ligand comprised only one N-heterocyclic aromatic group as shown in FIGS. 2-5 in the dimerization of ethylene to produce butenes.

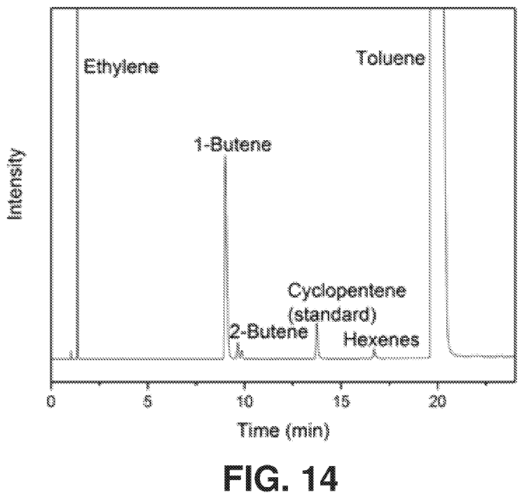

In a typical run, dried toluene, the catalyst and a co-catalyst (methyl aluminoxane (MAO)) were all added and stirred in a tank reactor that was kept at about 35° C. The amount of co-catalyst added was about 1000 equivalents of MAO. The ethylene was fed to the reactor with a total pressure of about 30 bar and kept there for about 10 minutes. Then, the reactor was quenched immediately in a mixture of dry ice and acetone in order to condense all products at a temperature of about −40° C. After that, cyclo-pentene was added as an external standard in order to analyze results in Gas Chromatography (As shown in FIG. 6 (before reaction) and FIG. 14 (after reaction)). KAUST-1/4 M-MAO effect ran with about 1000 equivalents of co-catalyst showed an activity of about 120,000 moles of ethylene consumed per moles of metal per hour (that was less active when the MAO equivalent was 2000 (table 1 for comparison)) with a very good selectivity towards butenes comprising about 95% of products while the other approx. 3% comprised hexenes. Analyzing the butenes product stream, about 90% of the butenes products were 1-butenes while the rest comprised a mixture of 2-butenes and iso-butene. The amount of co-catalyst therefore affected the reaction.

TABLE 1

| | Activity/TOF | Selectivity | Selectivity | P | T |
|---|---|---|---|---|---|
| Catalyst | (h⁻¹) | to C4 | to 1-Butene | (bar) | (° C.) |
| Commercial | 960,000 | 91.7 | 93 | 22-25 | 55 |
| Catalyst [2] | | | | | |
| KAUST-1M | 1,100,000 | 96 | 88 | 50 | 35 |
| KAUST-1/2M | 500,000 | 94 | 87 | 30 | 35 |
| KAUST-1/4M- | 250,000 | 94 | 87 | 30 | 35 |
| toulene washed | | | | | |
| KAUST-1/4M- | 170,000 | 95 | 90 | 30 | 35 |
| reused | | | | | |
| KAUST-1/4M- | 140,000 | 97 | 92 | 30 | 25 |
| temperature | | | | | |
| effect | | | | | |
| KAUST-1/4M | 250,000 | 95 | 89 | 30 | 35 |
| KAUST-1/4M- | 160,000 | 94 | 90 | 20 | 35 |
| pressure effect | | | | | |
| KAUST-1/4M- | 120,000 | 95 | 90 | 30 | 35 |
| MAO effect | | | | | |

TABLE 2

| | Activity/TOF | Selectivity | Selectivity | P | T |
|---|---|---|---|---|---|
| Catalyst | (h⁻¹) | to C4 | to 1-Butene | (bar) | (° C.) |
| KAUST-1M | 1,100,000 | 96 | 88 | 50 | 35 |
| KAUST-1/2M | 500,000 | 94 | 87.5 | 30 | 35 |
| KAUST-1/4M | 250,000 | 95 | 89.5 | 30 | 35 |
| Ni(1%)-mfu- | 41,500 | 97.4 | 94.5 | 50 | 25 |
| 4l [7] | | | | | |

### Example 10

### Supported Heterogeneous Catalysts

Below is one example illustrating a supported heterogeneous catalyst comprising a metal-organic framework.

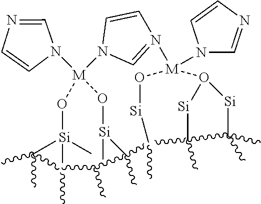

where M is one or more of zinc, titanium, nickel, copper, chromium, vanadium, cadmium, ruthenium, manganese, iron, cobalt, zirconium, silver, gold, magnesium, palladium, platinum, mercury, lead, aluminum, scandium, indium, and gallium.

### Example 11

### Preparation of Catalyst for Ethylene Oligomerization

Linear Alpha Olefins (LAO) are one of the most important products in the chemical industry with a market size of USD 8.26 billion in 2018 and ethylene is the most common feedstock to produce linear alpha olefins. Currently, there are two categories of linear alpha olefins processes where ethylene is used as a feedstock. Namely, on-purpose linear alpha olefins processes and wide range linear alpha olefins processes. On-purpose processes produces principally C4 (1-butene), C6 (1-hexene), or C8 (1-octene) linear alpha olefins from ethylene via ethylene dimerization, trimerization and tetramerization, respectively. This process is employed mainly to make co-monomers used in polyethylene and polypropylene production. On the other hand, wide range linear alpha olefins processes tend to produce products ranging between C₄ to C₂₀₊ in order to make additional applications. For instance, C₆-C₁₀ alkenes are used for plasticizer production and C₁₂-C₁₈ are demanded to produce detergent-range alcohols in order to be used in processes to make final products such as cosmetics and shampoos.

Currently, there are different commercialized technologies to produce wide range of LAO products. All commercialized technologies employ homogenous catalyst systems to convert ethylene to wide range of linear alpha olefins products. These homogenous systems tend to have very high catalyst recovery costs as the products and catalysts are in the same phase. Moreover, homogenous catalysis are a non-environmentally friendly option as it uses excessively large amounts of organic solvents. Heterogenous catalyst systems are options to overcome these deficiencies in homogenous catalysts. Accordingly, a heterogenous catalyst is provided in this Example that can produce wide range of linear alpha olefins (C₄-C₂₀₊) from ethylene where the catalyst can be separated, and the costly catalyst/product separation process is no longer needed.

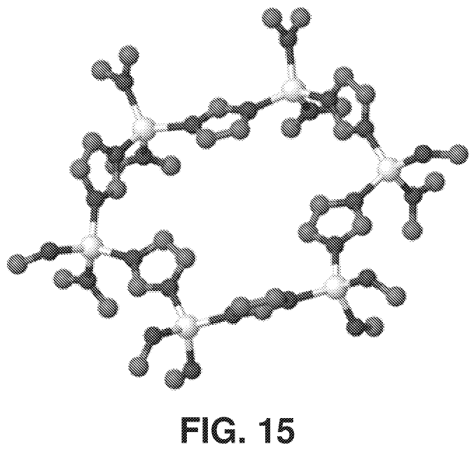

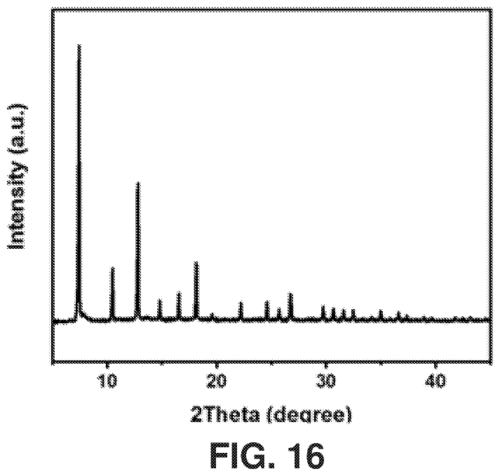

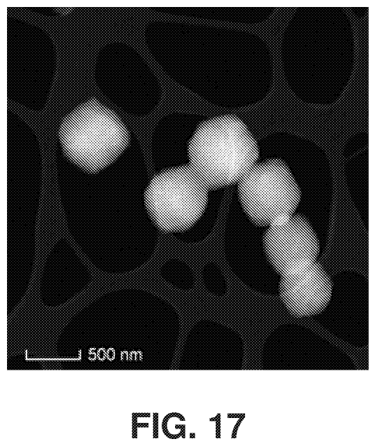

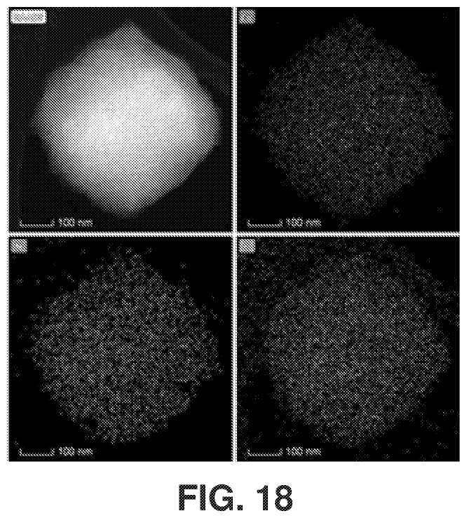

An exemplary metal doped-MOF as a heterogeneous catalyst for ethylene oligomerization using ligands having only one N-heterocyclic group (e.g., imidazole). For preparation of KAUST-OLI-101, a mixture of Co(NO₃)₂.6H₂O was dissolved in methanol (solution 1). Another solution (solution 2) consisting of methanol and 2-methylimidazole (FIG. 15) was mixed with solution 1 under stirring at room temperature. The mixed solution reacted for a duration, before it was centrifuged and washed with methanol several times. The modeled structure, PXRD pattern, Transmission Electron Microscopy Image Energy Dispersive X-ray Spectroscopy images are shown in FIGS. 15-18.

### Example 12

### Catalytic Performance of KAUST-OLI-101

The following Example shows the catalytic performance of a heterogeneous catalyst (KAUST-OLI-101) comprising a metal-doped metal-organic framework, wherein the metal-doped metal-organic framework includes ligands having only one N-heterocyclic aromatic group as shown in FIGS. 15-18 in the oligomerization of ethylene to produce wide range products of linear alpha olefins.

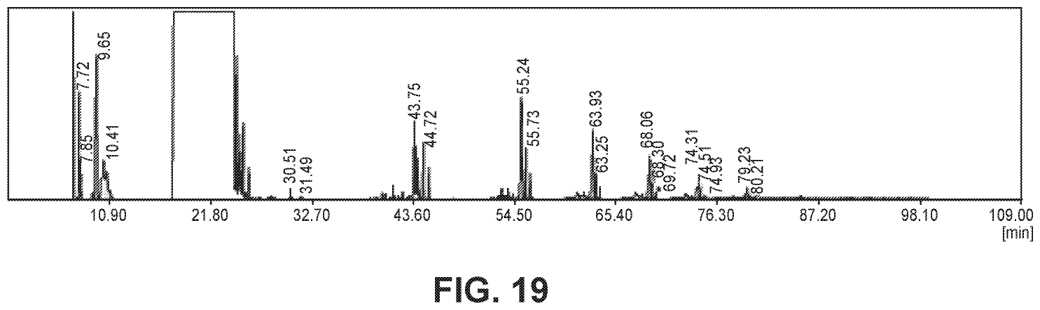

In a typical run, toluene, the catalyst, and a co-catalyst (methyl aluminoxane (MAO)) were added to a tank reactor kept at about 35° C. and stirred. The amount of co-catalyst added was about 1 ml of MAO (about 10 wt. % Al in toluene). The ethylene was fed to the reactor with a total pressure of 30 bar and kept there for 10 minutes. Then, the reactor was quenched immediately in a mixture of dry ice and acetone in order to condense all products at a temperature of about −40° C. Thereafter, cyclo-pentene was added as an external standard in order to analyze results in Gas Chromatography (As shown in FIG. 19). KAUST-OLI-101 catalyst showed a remarkable activity of 100 grams of products per gram of catalyst per hour with a very good overall selectivity towards linear alpha olefins comprising 65%. Analyzed product streams are compared with some current industrial processes and shown in below table:

| | | | Selectivity | Selectivity to | Selectivity | Selectivity |
|---|---|---|---|---|---|---|
| | Pressure | Temperature | to C4 | to C5-C10 | C12-C18 | to C20+ |
| Catalyst | (bar) | (° C.) | (wt. %) | (wt. %) | (wt. %) | (wt. %) |
| KAUST-OLI-101 | 30 | 35 | 11 | 42 | 45 | 2 |
| (heterogenous) | | | | | | |
| CPChem | 138-276 | 175-290 | 14 | 41 | 31 | 14 |
| (homogenous) ^{[4]} | | | | | | |
| Shell | 69-138 | 80-120 | 7-14 | 25-41 | 26-33 | 14-42 |
| (homogenous) ^{[4]} | | | | | | |

### Example 13

### Preparation of Catalyst for Propylene Dimerization

Propylene dimers are usually products of interest as it can be used as fuel additives and as a starting materials for the preparation of monomers. These C6 dimers include, for example and without limitation, methyl-pentenes, dimethyl-butenes, and n-hexenes. Among examples of products produced by one of these C6 dimers monomers is polymethylpenetene. It has been used for applications such as high pressure rubber hoses, transparent plastics with high melting point used in microwavable cookware and mold cups. 4-methyl-1-penetne, the monomer used for making polymethylpentene, is currently only produced via propylene dimerization. Another important C6 dimer is 2-methyl-1-pentene that can be used as an intermediate for the synthesis of isoprene. Moreover, a very special dimer is 2,3-dimethylbutene that is valuable as a gasoline additive with high octane number and low Reid vapor pressure.

Currently, the only commercialized technology for propylene dimerization uses homogenous catalyst complex systems to convert propylene to C6 products. These homogenous systems tend to have very high catalyst recovery costs as the products and catalysts are in the same phase. Moreover, homogenous catalysis are known as a non-environmentally friendly option as it uses excessively large amounts of organic solvents. Heterogenous catalyst systems are options to overcome these deficiencies in homogenous catalysts. Accordingly, the present Example provides a heterogenous catalyst that can produce C6 products from propylene with very high selectivity taking the advantages of catalyst recycle, very high activity and the costly catalyst/product separation process is no longer needed.

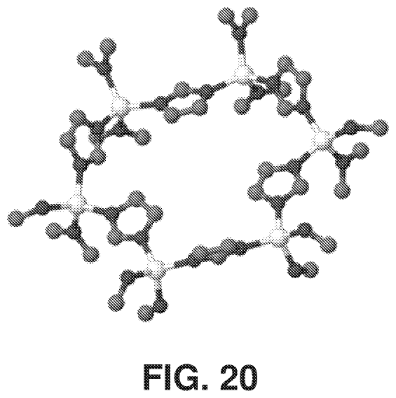

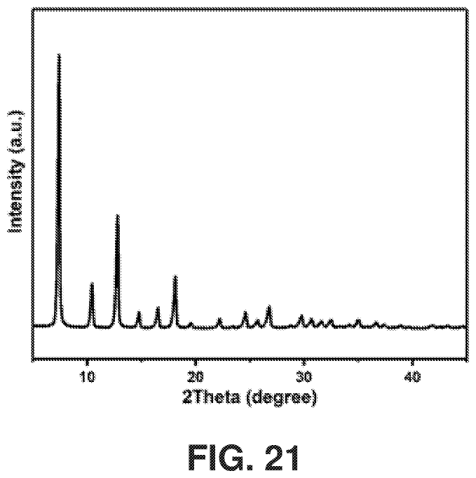

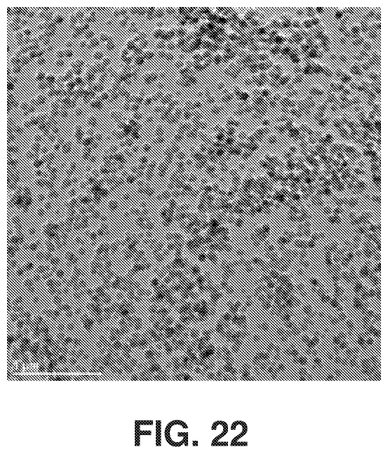

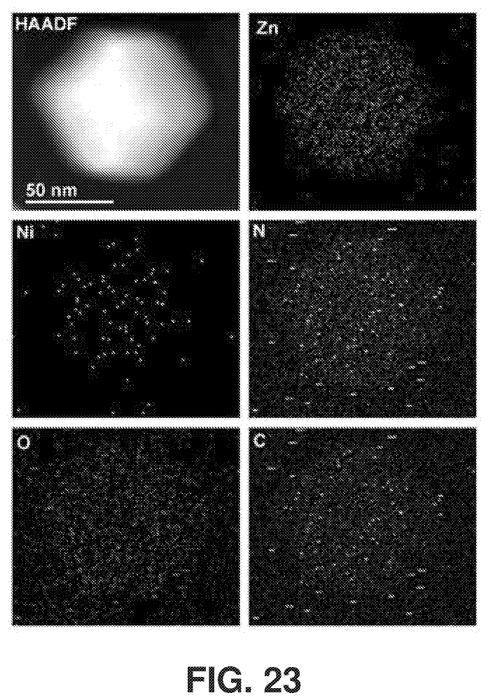

An exemplary metal doped-MOF as a heterogeneous catalyst for propylene dimerization using ligands having only one N-heterocyclic group (e.g., imidazole). For preparation of KAUST-Pro-101, a mixture of Zn(NO₃)₂.6H₂O and Ni(NO₃)₂. 6H₂O were dissolved in methanol (solution 1). Another solution (solution 2) consisting of methanol and 2-methylimidazole (FIG. 20) was mixed with solution 1 under stirring at room temperature. The mixed solution reacted for a duration, before it was centrifuged and washed with methanol several times. The modeled structure, PXRD pattern, Transmission Electron Microscopy Image Energy Dispersive X-ray Spectroscopy images are shown in FIGS. 20-23.

### Example 14

### Catalytic Performance of Kaust-Pro-101

The following Example shows the catalytic performance of a heterogeneous catalyst (KAUST-Pro-101) comprising a metal-doped metal-organic framework, wherein the metal-doped metal-organic framework includes ligands having only one N-heterocyclic aromatic group as shown in FIGS. 20-23 in the dimerization of propylene to produce C₆ dimers.

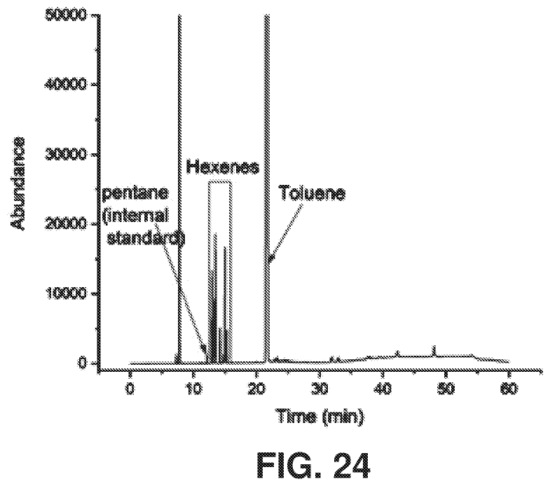

In a typical run, toluene, the catalyst, and a co-catalyst (methyl aluminoxane (MAO)) were added to a tank reactor kept at 35° C. and stirred. The amount of co-catalyst added was about 1 ml of MAO (about 10 wt. % Al in toluene). The propylene was fed to the reactor with a total pressure of 6.8 bar and kept there for 10 minutes. Then, the reactor was quenched immediately in a mixture of dry ice and acetone in order to condense all products at a temperature of −40° C. Thereafter, cyclo-pentene is added as an external standard in order to analyze results in Gas Chromatography (as shown in FIG. 24). KAUST-Pro-101 catalyst showed a remarkable activity of 2700 grams of products per gram of catalyst per hour with a very good overall selectivity towards C₆ products comprising 97%. Analyzed product stream are compared with some homogenous catalytic systems and shown in below table:

| | | | Activity | Selec- | Selec- |
|---|---|---|---|---|---|
| | | Temper- | (g-product/ | tivity | tivity to |
| | Pressure | ature | g-catalyst · | to C6 | others |
| Catalyst | (bar) | (C. °) | hr) | (wt. %) | (wt. %) |
| KAUST-Pro-101 | 6.8 | 35 | 2700 | 97 | 3 |
| (heterogenous) | | | | | |
| Hafnocene | 2 | 50 | 1800 | 34.3 | 65.7 |
| catalyst | | | | | |
| (3, Me, Me, Hf) | | | | | |
| (homogenous) | | | | | |
| [S] for | | | | | |
| comparison | | | | | |

Other embodiments of the present disclosure are possible. Although the description above contains much specificity, these should not be construed as limiting the scope of the disclosure, but as merely providing illustrations of some of the presently preferred embodiments of this disclosure. It is also contemplated that various combinations or sub-combinations of the specific features and aspects of the embodiments may be made and still fall within the scope of this disclosure. It should be understood that various features and aspects of the disclosed embodiments can be combined with or substituted for one another in order to form various embodiments. Thus, it is intended that the scope of at least some of the present disclosure should not be limited by the particular disclosed embodiments described above.

Thus the scope of this disclosure should be determined by the appended claims and their legal equivalents. Therefore, it will be appreciated that the scope of the present disclosure fully encompasses other embodiments which may become obvious to those skilled in the art, and that the scope of the present disclosure is accordingly to be limited by nothing other than the appended claims, in which reference to an element in the singular is not intended to mean “one and only one” unless explicitly so stated, but rather “one or more.” All structural, chemical, and functional equivalents to the elements of the above-described preferred embodiment that are known to those of ordinary skill in the art are expressly incorporated herein by reference and are intended to be encompassed by the present claims. Moreover, it is not necessary for a device or method to address each and every problem sought to be solved by the present disclosure, for it to be encompassed by the present claims. Furthermore, no element, component, or method step in the present disclosure is intended to be dedicated to the public regardless of whether the element, component, or method step is explicitly recited in the claims.

The foregoing description of various preferred embodiments of the disclosure have been presented for purposes of illustration and description. It is not intended to be exhaustive or to limit the disclosure to the precise embodiments, and obviously many modifications and variations are possible in light of the above teaching. The example embodiments, as described above, were chosen and described in order to best explain the principles of the disclosure and its practical application to thereby enable others skilled in the art to best utilize the disclosure in various embodiments and with various modifications as are suited to the particular use contemplated. It is intended that the scope of the disclosure be defined by the claims appended hereto

Various examples have been described. These and other examples are within the scope of the following claims.

What is claimed is:

1. A catalyst composition comprising:
a heterogeneous oligomerization catalyst including a metal-organic framework, the metal-organic framework including a plurality of first metal ions coordinated to one or more ligands, wherein each of the one or more ligands has only one N-heterocyclic aromatic group.

2. The catalyst composition of claim 1, wherein the plurality of first metal ions includes one of Zn, Ti, Ni, Cu, Cr, V, Cd, Co, Zr, Fe, Al, Mn, and Ru.

3. The catalyst composition of claim 1, wherein the plurality of first metal ions includes one of Ti, Ni, Cu, Co, Cr, and V.

4. The catalyst composition of claim 1, wherein the plurality of first metal ions includes one of Co, Ni, and Zn.

5. The catalyst composition of claim 1, wherein the first metal ion content is in the range of about 0.01% to about 60% by weight of the heterogeneous oligomerization catalyst.

6. The catalyst composition of claim 1, wherein the plurality of first metal ions are coordinated to each ligand through a nitrogen heteroatom of the N-heterocyclic aromatic group, or a coordinating O- or S-donor moiety of the ligand.

7. The catalyst composition of claim 1, wherein the one or more ligands of the metal-organic framework consist of ligands having only one N-heterocyclic group.

8. The catalyst composition of claim 1, wherein the one or more ligands of the metal-organic framework have the chemical structure of formula (I): 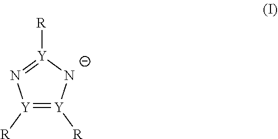
wherein:
each Y is independently N or C; and
each R is independently nothing, a hydrogen, an alkyl, or a counterion.

9. The catalyst composition of claim 1, wherein the one or more ligands of the metal-organic framework have the chemical structure of formula (II): 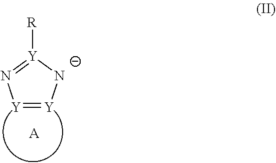
wherein:
each Y is independently N or C;
each R is independently nothing, a hydrogen, an alkyl, or a counterion; and
A is an aromatic or aliphatic group, optionally without any coordinating sites.

10. The catalyst composition of claim 1, wherein the one or more ligands includes one of pyrrolate, pyrazolate, triazolate, imidazolate, oxazolate, tetrazolate, pyridinate, thiazolate, oxadiazolate, purinate, quinolonate, and indolate.

11. The catalyst composition of claim 1, wherein the one or more ligands includes one of imidazolate, triazolate, and tetrazolate.

12. The catalyst composition of claim 1, wherein the one or more ligands has the formula: R^{a}—(N-heterocyclic aromatic group)-R^{b}, wherein R^{a} and R^{b} are the same or different and wherein each of R^{a} and R^{b} is independently —H, -alkyl, —NO₂, —R′, —F, —Cl, —Br, —I, —CN, —NC, —SO₃R′, —SO₃H, —OR′, —OH, —SR′, —SH, — PO₃R′, —PO₃H, —CF₃, —NR′₂, —NHR′, and —NH₂, wherein each R′ is the same or different and wherein each R′ is optionally substituted alkyl or optionally substituted aryl, provided that R′ is not an N-heterocyclic aryl.

13. The catalyst composition of claim 1, wherein the heterogeneous catalyst further includes a metal dopant.

14. The catalyst composition of claim 13, wherein the metal dopant includes a plurality of second metal ions and wherein the plurality of second metal ions includes one of Ti, Ni, Cu, Cr, Co, Zr, Fe, Al, Mn, Ru, Pd, Pt, and V.

15. The catalyst composition of claim 14, wherein the plurality of second metal ions are coordinated to each ligand through a nitrogen heteroatom of the N-heterocyclic aromatic group, or a coordinating O- or S-donor moiety of the ligand.

16. The catalyst composition of claim 14, wherein the second metal ion content is in the range of about 0% to about 60% by weight of the heterogeneous catalyst.

17. The catalyst composition of claim 1, further comprising a support, wherein the heterogeneous oligomerization catalyst has the chemical structure: 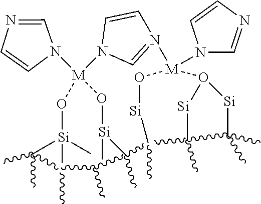
where M is one or more of zinc, titanium, nickel, copper, chromium, vanadium, cadmium, ruthenium, manganese, iron, cobalt, zirconium, silver, gold, magnesium, palladium, platinum, mercury, lead, aluminum, scandium, indium, and gallium.

18. A method of oligomerization comprising:
contacting one or more olefins with the heterogeneous oligomerization catalyst of claim 1 to form one or more oligomers.

19. The method of oligomerization of claim 18, wherein the one or more olefins include one or more of ethylene and propylene.

20. The method of claim 19, wherein the one or more oligomers include one or more butenes, one or more linear alpha olefins, and/or one or more C₆ dimers.
